(19) United States
(12) Patent Application Publication
(10) Pub. No.: US 2018/0058437 A1
(43) Pub. Date: Mar. 1, 2018

(54) PUMP WITH EXTERNAL CONTROLLED COMPRESSION AND METHODS OF PUMPING WITH EXTERNAL CONTROLLED COMPRESSION

(71) Applicant: THE REGENTS OF THE UNIVERSITY OF COLORADO, A BODY CORPORATE, Denver, CO (US)

(72) Inventors: Derek Eilers, Denver, CO (US); James Jaggers, Denver, CO (US); Robin Shandas, Boulder, CO (US); Scott Lawson, Denver, CO (US)

(21) Appl. No.: 15/551,521

(22) PCT Filed: Feb. 16, 2016
(86) PCT No.: PCT/US16/18091

§ 371 Date: Aug. 16, 2017

Related U.S. Application Data
(60) Provisional application No. 62/116,839, filed on Feb. 16, 2015.

(51) Int. Cl. F04B 43/12; A61M 1/10; A61M 1/12; C12M 1/00

(52) U.S. Cl. CPC F04B 43/123; A61M 1/1041; F04B 53/18; C12M 29/14; A61M 1/122

(57) ABSTRACT

The present invention relates to pumps, particularly a compact, linear, positive displacement pump that can be used in several applications including medical and non-medical devices. In particular, the invention is related to linear, positive displacement compression blood pumps (either intracorporeal or extracorporeal) that provide systemic circulatory stability by maintaining a steady average blood pressure. In some embodiments, the invention relates to perfusion pumps, infusion pumps, pumps used for ECMO (extracorporeal membrane oxygenators) and bioreactor pumps.

### CROSS-REFERENCE TO RELATED APPLICATIONS

The present application claims the benefit of U.S. Provisional Patent Application No. 62/116,839, filed on. Feb. 16, 2015, which is incorporated herein by reference.

### FIELD OF THE INVENTION

The present invention relates to pumps, particularly a compact, linear, positive displacement pump that can be used in several applications including medical and non-medical devices. In particular, the invention is related to linear, positive displacement compression blood pumps (either intracorporeal or extracorporeal) that provide systemic circulatory stability by maintaining a steady average blood pressure. In some embodiments, the invention relates to perfusion pumps, infusion pumps, pumps used for ECMO (extracorporeal membrane oxygenators) and bioreactor pumps.

### BACKGROUND OF THE INVENTION

Generally, pumps operate with different types of actuation and present challenges with respect to speed, size, and flow. For medical and research applications, there remains a need to decrease risks to the patients or living system by minimizing part contact with blood, bodily fluids, growth medium or other fluid surrounding a living system when involving pumping of biologically related fluids or fluids into a patient or living system.

### SUMMARY OF THE INVENTION

The present invention relates to pumps, particularly a compact, linear, positive displacement pump that can be used in several applications including medical and non-medical devices. In particular, the invention is related to linear, positive displacement compression blood pumps (either intracorporeal or extracorporeal) that provide systemic circulatory stability by maintaining a steady average blood pressure. In some embodiments, the invention relates to perfusion pumps, infusion pumps, pumps used for ECMO (extracorporeal membrane oxygenators) and bioreactor pumps.

In one embodiment, the present invention contemplates an external cam controlled compression pump, comprising a tube having a first end and a second end in opposition to one another, and a sidewall surrounding a lumen extending between the first end and the second end; a cam having a first end and a second end in opposition to one another, a tube contact surface disposed between the first end and the second end; a mount portion configured to rotatably support the cam and compressively position the tube contact surface of the cam in contact with the sidewall of the tube; and a motor in operable connection with the cam and configured to rotate the cam to drive the tube contact surface along the sidewall of the tube from a first location adjacent the first end of the tube to a second location adjacent the second end of the tube.

In one embodiment, the present invention contemplates a method comprising: a) providing; i) an external cam controlled compression pump; and ii) at least one cardiovascular vessel, said vessel comprising fluid; b) positioning a tube contact surface of a cam in contact with a sidewall of at least one tube, wherein said tube is connected to said at least one cardiovascular vessel; c) rotating the cam in contact with the sidewall of the tube to reposition the tube contact surface from a first location adjacent the first end of the tube to a second location adjacent a second end of the tube, and d) pumping said fluid through said at least one cardiovascular vessel. In one embodiment, said fluid comprises blood. In one embodiment, said fluid is selected from the group consisting of nutrients or thereapeutic agents (medications)—such as insulin or other hormones, antibiotics, chemotherapy drugs, and pain relievers. In one embodiment, said cam varies in length. In one embodiment, said cam comprises a helically shaped ridge or raised section that is supported through its center by a cylindrical section that it wraps around. In one embodiment, said cam comprises a helical ridge around a form with at least one axis or rotation. In one embodiment, said helical ridge comprises said tube contact surface of said cam. In one embodiment, said cam comprises a helix or spiral. In one embodiment, said helix wraps more than 360 degrees around an axis of rotation. In one embodiment, said helix wraps less than 360 degrees around an axis of rotation. In one embodiment, said axis of rotation comprises a central support shaft. In one embodiment, said cam comprises a tapered end. In one embodiment, said helical ridge comprises a solid with a radius. In one embodiment, said helical ridge comprises a solid without a radius. In one embodiment, said ridge comprises an attachment feature. In one embodiment, said attachement feature is a friction-reducing element. In one embodiment, said ridge comprises ball bearings. In one embodiment, said ridge comprises rollers. In one embodiment, said friction-reducing element comprises lubricant. In one embodiment, said friction-reducing element comprises a lubricant reservoir. In one embodiment, said friction-reducing element comprises ball bearings. In one embodiment, said friction-reducing element comprises rollers. In one embodiment, said cam comprises plastic. In one embodiment, said cam comprises metallic alloys. In one embodiment, said cam comprises composite material. In one embodiment, said cam comprises one solid center shaft and helical ridge. In one embodiment, said cam comprises several linked segments with helical ridges. In one embodiment, said pump comprises more than one tube. In one embodiment, multiple tubes are used with one cam. In one embodiment, said tubes are constrained around the perimeter of the cam, parallel to the cam rotational axis, with a case to properly constrain said tubes. In one embodiment, said tube comprises a tube with extended legs. In one embodiment, said tube comprises standard medical grade tubing.

In one embodiment, the present invention contemplates a method comprising: a) providing; i) an external cam controlled compression pump; ii) a subject comprising at least one first vessel, said vessel comprising fluid; iii) at least one second vessel comprising second fluid for delivery, and iv) at least one tube comprising a first and second end; b) positioning a tube contact surface of a cam in contact with a sidewall of at least one tube, wherein said tube first end is connected to said at least one first vessel and said tube second end is connected to said at least one second vessel; b) rotating the cam in contact with the sidewall of the tube to reposition the tube contact surface from a first location adjacent the first end of the tube to a second location adjacent a second end of the tube, and c) pumping said fluid through said at least one vessel. In one embodiment, said first vessel comprises a cardiovascular vessel. In one embodiment, said first vessel comprises a feeding tube. In one embodiment, said first vessel comprises a line to the central nervous system. In one embodiment, said first vessel comprises a line to a subcutaneous entry point. In one embodiment, said fluid comprises blood. In one embodiment, said second fluid for delivery comprises blood. In one embodiment, said second fluid for delivery is selected from the group consisting of nutrients or thereapeutic agents (medications). In one embodiment, said thereapeutic agents include insulin, other hormones, antibiotics, chemotherapy drugs, and pain relievers. In one embodiment, said pumping treats at least one symptom of said subject. In one embodiment, said cam varies in length. In one embodiment, said cam comprises a helically shaped ridge or raised section that is supported through its center by a cylindrical section that it wraps around. In one embodiment, said cam comprises a helical ridge around a form with at least one axis or rotation. In one embodiment, said helical ridge comprises said tube contact surface of said cam. In one embodiment, said cam comprises a helix. In one embodiment, said helix wraps more than 360 degrees around an axis of rotation. In one embodiment, said helix wraps less than 360 degrees around an axis of rotation. In one embodiment, said axis of rotation comprises a central support shaft. In one embodiment, said cam comprises a tapered end. In one embodiment, said helical ridge comprises a solid with a radius. In one embodiment, said helical ridge comprises a solid without a radius. In one embodiment, said ridge comprises an attachment feature. In one embodiment, said attachement feature is a friction-reducing element. In one embodiment, said ridge comprises ball bearings. In one embodiment, said ridge comprises rollers. In one embodiment, said friction-reducing element comprises lubricant. In one embodiment, said friction-reducing element comprises a lubricant reservoir. In one embodiment, said friction-reducing element comprises ball bearings. In one embodiment, said friction-reducing element comprises rollers. In one embodiment, said cam comprises plastic. In one embodiment, said cam comprises metallic alloys. In one embodiment, said cam comprises composite material. In one embodiment, said cam comprises one solid center shaft and helical ridge. In one embodiment, said cam comprises several linked segments with helical ridges. In one embodiment, said pump comprises more than one tube. In one embodiment, multiple tubes are used with one cam. In one embodiment, said tubes are constrained around the perimeter of the cam, parallel to the cam rotational axis, with a case to properly constrain said tubes. In one embodiment, said tube comprises a tube with extended legs. In one embodiment, said tube comprises standard medical grade tubing.

In one embodiment, the invention contemplates an external cam controlled compression pump comprising: a) at least one tube having a first end and a second end in opposition to one another, and a sidewall surrounding a lumen extending between the first end and the second end; b) a cam having a first end and a second end in opposition to one another, a tube contact surface disposed between the first end and the second end; c) a mount portion configured to rotatably support the cam and compressively position the tube contact surface of the cam in contact with the sidewall of the tube; and d) a motor in operable connection with the cam and configured to rotate the cam to drive the tube contact surface along the sidewall of the tube from a first location adjacent the first end of the tube to a second location adjacent the second end of the tube. In one embodiment, the tube contact surface of the cam is outside of the sidewall of the tube. In one embodiment, said pump is configured as a device for augmenting blood flow in a single ventricle circuit. In one embodiment, said pump further comprising a biocompatible, implantable housing in a surrounding configuration to the tube, cam, and motor. In one embodiment, said pump further comprising a biocompatible, implantable housing for intracorporeal implantation. In one embodiment, the cam is configured to provide chamber compression within the tube. In one embodiment, said pump further comprising an inflow cannula and an outflow cannula in attachment to the first end and the second end of the tube, respectively. In one embodiment, said pump further comprising a housing for extracorporeal positioning. In one embodiment, said tube is flexible. In one embodiment, said pump further comprising a constraint portion in connection with the flexible chamber, the constraint portion providing anchoring with the mount portion. In one embodiment, said cam is integral to a shaft of the motor. In one embodiment, said pump is configured to perform as at least one of an infusion pump, an oxygenator, a heat exchanger, and a venous reservoir. In one embodiment, said pump is configured to perform as implantable infusion pump. In one embodiment, said pump further comprises at least one of an infusion pump, an oxygenator, a heat exchanger, and a venous reservoir. In one embodiment, said pump is configured to perform as a perfusion circuit. In one embodiment, said pump is configured to perform as a vein dilator for AV fistulas. In one embodiment, said pump is configured to perform as on-medical applications needing pulsatile supporting flow. In one embodiment, said pump is configured to perform as on-medical applications needing continuous supporting flow. In one embodiment, an axis of rotation of the cam is parallel to a longitudinal axis of the tube. In one embodiment, the tube, the cam, and the motor are operable in an in-line flow configuration so as to reduce a length of tubing to and from a patient. In one embodiment, said pump comprises a perfusion pump. In one embodiment, said pump comprises an infusion pump. In one embodiment, said pump comprises a ventricular assist device. In one embodiment, said pump comprises an extracorporeal membrane oxygenator. In one embodiment, said cam varies in length. In one embodiment, said cam comprises a helically shaped ridge or raised section that is supported through its center by a cylindrical section that it wraps around. In one embodiment, said cam comprises a helical ridge around a form with at least one axis or rotation. In one embodiment, said helical ridge comprises said tube contact surface of said cam. In one embodiment, said cam comprises a helix. In one embodiment, said helix wraps more than 360 degrees around an axis of rotation. In one embodiment, said helix wraps less than 360 degrees around an axis of rotation. In one embodiment, said axis of rotation comprises a central support shaft. In one embodiment, said cam comprises a tapered end. In one embodiment, said helical ridge comprises a solid with a radius. In one embodiment, said helical ridge comprises a solid without a radius. In one embodiment, said ridge comprises an attachment feature. In one embodiment, said attachement feature is a friction-reducing element. In one embodiment, said ridge comprises ball bearings. In one embodiment, said ridge comprises rollers. In one embodiment, said friction-reducing element comprises lubricant. In one embodiment, said friction-reducing element comprises a lubricant reservoir. In one embodiment, said friction-reducing element comprises ball bearings. In one embodiment, said friction-reducing element comprises rollers. In one embodiment, said cam comprises plastic. In one embodiment, said cam comprises metallic alloys. In one embodiment, said cam comprises composite material. In one embodiment, said cam comprises one solid center shaft and helical ridge. In one embodiment, said cam comprises several linked segments with helical ridges. In one embodiment, said pump comprises more than one tube. In one embodiment, multiple tubes are used with one cam. In one embodiment, said tubes are constrained around the perimeter of the cam, parallel to the cam rotational axis, with a case to properly constrain said tubes. In one embodiment, said tube comprises a tube with extended legs. In one embodiment, said tube comprises standard medical grade tubing.

In one embodiment, the invention contemplates a method comprising: a) providing: i) an external cam controlled compression pump, said pump comprising a motor and a cam having a tube contact surface and a tube sidewall; and ii) at least one cardiovascular vessel, said vessel comprising blood a) positioning said tube contact surface of said cam in contact with said tube sidewall, wherein said tube is connected to said at least one cardiovascular vessel; b) rotating the cam in contact with the tube sidewall to reposition the tube contact surface from a first location adjacent the first end of the tube to a second location adjacent a second end of the tube; and c) pumping said blood through said at least one cardiovascular vessel. In one embodiment, the tube, the cam, and the motor are operable to provide occlusion adjustment. In one embodiment, an axis of rotation of the cam is parallel to a longitudinal axis of the tube. In one embodiment, said method further comprising using multiple cams that are placed on adjacent sides of the tubes to squeeze together the tube. In one embodiment, the multiple cams are geared to run off a single motor. In one embodiment, the multiple cams are geared to run off multiple motors. In one embodiment, the multiple cams have lobes offset to one another. In one embodiment, the multiple cams have lobes directly parallel to one another. In one embodiment, said pump comprises a perfusion pump. In one embodiment, said pump comprises an infusion pump. In one embodiment, said pump comprises a ventricular assist device. In one embodiment, said cam varies in length. In one embodiment, said cam comprises a helically shaped ridge or raised section that is supported through its center by a cylindrical section that it wraps around. In one embodiment, said cam comprises a helical ridge around a form with at least one axis or rotation. In one embodiment, said helical ridge comprises said tube contact surface of said cam. In one embodiment, said cam comprises a helix. In one embodiment, said helix wraps more than 360 degrees around an axis of rotation. In one embodiment, said helix wraps less than 360 degrees around an axis of rotation. In one embodiment, said axis of rotation comprises a central support shaft. In one embodiment, said cam comprises a tapered end. In one embodiment, said helical ridge comprises a solid with a radius. In one embodiment, said helical ridge comprises a solid without a radius. In one embodiment, said ridge comprises an attachment feature. In one embodiment, said attachement feature is a friction-reducing element. In one embodiment, said ridge comprises ball bearings. In one embodiment, said ridge comprises rollers. In one embodiment, said friction-reducing element comprises lubricant. In one embodiment, said friction-reducing element comprises a lubricant reservoir. In one embodiment, said friction-reducing element comprises ball bearings. In one embodiment, said friction-reducing element comprises rollers. In one embodiment, said cam comprises plastic. In one embodiment, said cam comprises metallic alloys. In one embodiment, said cam comprises composite material. In one embodiment, said cam comprises one solid center shaft and helical ridge. In one embodiment, said cam comprises several linked segments with helical ridges. In one embodiment, said pump comprises more than one tube. In one embodiment, multiple tubes are used with one cam. In one embodiment, said tubes are constrained around the perimeter of the cam, parallel to the cam rotational axis, with a case to properly constrain said tubes. In one embodiment, said tube comprises a tube with extended legs. In one embodiment, said tube comprises standard medical grade tubing.

In one embodiment, the invention contemplates an external cam controlled compression pump comprising: a) at least one tube having a first end and a second end in opposition to one another, and a sidewall surrounding a lumen extending between the first end and the second end; b) a cam having a first end and a second end in opposition to one another, c) at least one tube contact surface disposed between the first end and the second end of said tube; d) a mount portion configured to rotatably support the cam and compressively position the tube contact surface of the cam in contact with the sidewall of the tube; e) a motor in operable connection with the cam and configured to rotate the cam to drive the tube contact surface along the sidewall of the tube from a first location adjacent the first end of the tube to a second location adjacent the second end of the tube; and f) a base configured to constrain the tube with a geometry that matches a profile of the cam so as to maximize occlusion. In one embodiment, said base is configured to constrain the tube in a manner that limits the lateral motion of the tube as it is compressed by the cam. In one embodiment, said pump further comprises multiple tubes together with a single one of the cam configured to operate with one another, wherein the tubes are constrained around the perimeter of the cam, parallel to the cam rotational axis, and a case constrains the multiple tubes. In one embodiment, said pump comprises a perfusion pump. In one embodiment, said pump comprises an infusion pump. In one embodiment, said pump comprises a ventricular assist device. In one embodiment, said pump comprises a bioreactor pump. In one embodiment, said pump comprises an extracorporeal membrane oxygenator. In one embodiment, said cam varies in length. In one embodiment, said cam comprises a helically shaped ridge or raised section that is supported through its center by a cylindrical section that it wraps around. In one embodiment, said cam comprises a helical ridge around a form with at least one axis or rotation. In one embodiment, said helical ridge comprises said tube contact surface of said cam. In one embodiment, said cam comprises a helix. In one embodiment, said helix wraps more than 360 degrees around an axis of rotation. In one embodiment, said helix wraps less than 360 degrees around an axis of rotation. In one embodiment, said axis of rotation comprises a central support shaft. In one embodiment, said cam comprises a tapered end. In one embodiment, said helical ridge comprises a solid with a radius. In one embodiment, said helical ridge comprises a solid without a radius. In one embodiment, said ridge comprises an attachment feature. In one embodiment, said attachement feature is a friction-reducing element. In one embodiment, said ridge comprises ball bearings. In one embodiment, said ridge comprises rollers. In one embodiment, said friction-reducing element comprises lubricant. In one embodiment, said friction-reducing element comprises a lubricant reservoir. In one embodiment, said friction-reducing element comprises ball bearings. In one embodiment, said friction-reducing element comprises rollers. In one embodiment, said cam comprises plastic. In one embodiment, said cam comprises metallic alloys. In one embodiment, said cam comprises composite material. In one embodiment, said cam comprises one solid center shaft and helical ridge. In one embodiment, said cam comprises several linked segments with helical ridges. In one embodiment, said pump comprises more than one tube. In one embodiment, multiple tubes are used with one cam. In one embodiment, said tubes are constrained around the perimeter of the cam, parallel to the cam rotational axis, with a case to properly constrain said tubes. In one embodiment, said tube comprises a tube with extended legs. In one embodiment, said tube comprises standard medical grade tubing.

Other objects, features, and advantages of the invention will become apparent from the following detailed description of the invention with reference to the accompanying drawings.

### Definitions

The term “perfusion”, as used herein, refers to the process of a body delivering blood to a capillary bed in its biological tissue.

The terms “overperfusion” and “underperfusion” are measured relative to the average level of perfusion that exists across all the tissues in an individual body, and should not be confused with wrong hypoperfusion and “hyperperfusion”, which measure the perfusion level relative to a tissue's current need to meet its metabolic needs. Heart tissues, for example, are usually classified as being overperfused because they normally are receiving more blood than the rest of tissues in the organism. In the case of skin cells, extra blood flow in them is used for thermoregulation of a body. In addition to delivering oxygen, blood flow helps to dissipate heat in a physical body by redirecting warm blood closer to its surface where it can help to cool a body through sweating and thermal dissipation.

The term “perfusion pump”, as used herein, refers to a device for simulating cardiopulmonary function.

The term “perfusion circuit”, as used herein, refers to a perfusion system for the extracorporeal preservation of vitality or regeneration of organs, limbs or tissue lobes for use in transplant surgery, extracorporeal support of the liver, or for biochemical or pharmacological study of isolated organs. The system comprises an organ perfusion chamber filled with dialysate as the storage fluid and equipped with a temperature control device. An example of such a perfusion circuit is a cardiopulmonary bypass (CPB) machine or pump also known as a heart-lung machine. CPB is a form of extracorporeal circulation.

The term “infusion pump”, as used herein, refers to a medical device that delivers fluids, such as nutrients and medications, into a patient's body in controlled amounts. Infusion pumps are in widespread use in clinical settings such as hospitals, nursing homes, and in the home. An infusion pump infuses fluids, medication or nutrients into a patient's circulatory system. It is generally used intravenously, although subcutaneous, arterial and epidural infusions are occasionally used.

The term “bioreactor pump”, as used herein, refers to a pump used in a system designed to grow cells. In some embodiments, said cells are bacterial, fungal, or eukaryote. In some embodiments, said bioreactor pump is used to provide continuous flow of growth medium.

The term “fluid”, as used herein, refers to a fluid is a substance that continually deforms (flows) under an applied shear stress. In biological terms, a fluid is generally blood, a growth medium, lymph fluid, therapeutic agents in liquid, or other biological fluids. The present invention is not to be limited by the nature of the fluid. In some embodiments, fluid may comprise a plurality of small solid elements which may respond to pumping as a fluid. In some embodiments, a fluid may comprise a mixture of fluid and solid elements. In some embodiments, a fluid comprises viscous solutions, solid laden liquids, slurries or pastes.

The term “delivery vessel”, as used herein, refers to an avenue to delivery for a fluid into a subject or living system. In the case of an animal, such as an intravenous (IV) tube for a blood vessel, feeding tube, epidural tube to CNS system, etc. In the case of a living system, such as a cell culture system, this may be a series of channels, tubes, or lines which enable the continued flow of growth medium.

The term “cannula”, as used herein, refers to a tube that can be inserted into the body of a subject, often for the delivery or removal of fluid.

The term “cardiovascular vessel”, as used herein, refers to a vessel of the cardiovascular system.

The term “cardiovascular system”, as used herein, refers to an organ system that permits blood to circulate and transport nutrients (such as amino acids and electrolytes), oxygen, carbon dioxide, hormones, and blood cells to and from the cells in the body to provide nourishment and help in fighting diseases, stabilize temperature and pH, and maintain homeostasis.

The term “feeding tube”, as used herein, refers to a medical device used to provide nutrition to patients who cannot obtain nutrition by mouth, are unable to swallow safely, or need nutritional supplementation

The term “extracorporeal”, as used herein, refers to being situated outside the body, such as the body of a subject or the bounds of a living system.

The term “intracorporeal”, as used herein, refers to being situated inside the body, such as the body of a subject or the bounds of a living system.

The term “suspected of having”, as used herein, refers to a medical condition or set of medical conditions (e.g., preliminary symptoms) exhibited by a patient that is insufficient to provide a differential diagnosis. Nonetheless, the exhibited condition(s) would justify further testing (e.g., condition testing) to obtain further information on which to base a diagnosis.

The term “at risk for” as used herein, refers to a medical condition or set of medical conditions exhibited by a patient that may predispose the patient to a particular disease or affliction. For example, these conditions may result from influences that include, but are not limited to, physiological, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms).

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “pulmonary injury” as used herein, refers to any effect on pulmonary tissue that impairs its functional or structural integrity. For example, injury may be a result of, but not limited to, age, malformation, disease, inhalation of toxins, surgical procedures, or accident.

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc.

The term “medium” as used herein, refers to any material, or combination of materials, which serve as a carrier or vehicle for delivering of a drug to a treatment point (e.g., wound, surgical site etc.). For all practical purposes, therefore, the term “medium” is considered synonymous with the term “carrier”. It should be recognized by those having skill in the art that a medium comprises a carrier, wherein said carrier is attached to a drug or drug and said medium facilitates delivery of said carrier to a treatment point. Further, a carrier may comprise an attached drug wherein said carrier facilitates delivery of said drug to a treatment point. Preferably, a medium is selected from the group including, but not limited to, foams, gels (including, but not limited to, hydrogels), xerogels, microparticles (i.e., microspheres, liposomes, microcapsules etc.), bioadhesives, or liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of drugs over a period of time lasting approximately from 1 day to 6 months.

The term “drug” or “compound” or “therapeutic agent” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars. Non-limiting examples of therapeutic agents include: hormones, antibiotics, chemotherapy drugs, and pain relievers.

The term “administered” or “administering”, as used herein, refers to any method of providing a composition or therapeutic treatment to a patient such that the composition or therapeutic treatment has its intended effect on the patient. An exemplary method of administering is by a direct mechanism such as, local tissue administration (i.e., for example, extravascular placement), intravenous injection, intravenous line, oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatiblity is evaluated according to the application for which it was designed: for example; a bandage is regarded a biocompable with the skin, whereas an implanted medical device is regarded as biocompatible with the internal tissues of the body. Preferably, biocompatible materials include, but are not limited to, biodegradable and biostable materials.

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a compound or drug to a patient during a medical procedure or therapy is contemplated herein as a medical device. “Direct medical implants” include, but are not limited to, intracopreal pump, ventricular assist devices, infusion pump, feeding pump, extracorporeal pump, perfusion pumps, extracorporeal membrane oxygenators urinary and intravascular catheters, dialysis shunts, wound drain tubes, skin sutures, vascular grafts and implantable meshes, intraocular devices, implantable drug delivery systems and heart valves, and the like. “Wound care devices” include, but are not limited to, general wound dressings, non-adherent dressings, burn dressings, biological graft materials, tape closures and dressings, and surgical drapes. “Surgical devices” include, but are not limited to, intracopreal pump, ventricular assist devices, infusion pump, feeding pump, extracorporeal pump, perfusion pumps, extracorporeal membrane oxygenators, endoscope systems (i.e., catheters, vascular catheters, surgical tools such as scalpels, retractors, and the like) and temporary drug delivery devices such as infusion pumps, drug ports, injection needles etc. to administer the medium. A medical device is “coated” when a medium comprising a cytostatic or antiproliferative drug (i.e., for example, sirolimus or an analog of sirolimus) becomes attached to the surface of the medical device. This attachment may be permanent or temporary. When temporary, the attachment may result in a controlled release of a cytostatic or antiproliferative drug.

### BRIEF DESCRIPTION OF THE DRAWINGS

The drawings included in the present application are incorporated into, and form part of, the specification. They illustrate embodiments of the present invention and, along with the description, serve to explain the principles of the invention. The drawings are only illustrative of embodiments of the invention and do not limit the invention. Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 illustrates one embodiment of a compression pump as disclosed herein.

FIG. 2 illustrates one embodiment of a compression pump as disclosed herein having overall dimensions (excluding flat base portion and controller) of 35 mm Width×200 mm Length×88 mm Height.

FIG. 3 illustrates one embodiment of a cam shape (isometric and top views)

FIG. 4 illustrates a phase 2 prototype of one embodiment of an external compression pump which is similar to FIG. 2.

FIG. 5 illustrates one embodiment of a motor having a shaft for driving the cam of the pump.

FIG. 6 illustrates three potential cam shapes for the pump.

FIGS. 7A&B illustrates graphical data of pressure and flow plated over a time interval for one of the three cam shapes in FIG. 6.

FIG. 8 illustrates graphical data of input pressure and output pressure.

FIGS. 9A&B illustrates graphical data of pressure from the pump.

FIG. 10 illustrates an in vitro circulation set up of the cavopulmonary assist device.

FIG. 11 illustrates a cross-sectional view of an exemplary embodiment of a tube with attachment anchor portions.

FIG. 12 shows a cross section of a custom tube shape created using a silicone extrusion process.

FIG. 13 illustrates a view of a typical roller pump.

FIG. 14 illustrates one embodiment of a pump as disclosed herein having overall dimensions (excluding flat base portion) of 30 mm width×135 mm length×47 mm height.

FIG. 15 illustrates one embodiment of a pump as disclosed herein having overall dimensions (excluding flat base portion and controller) of 35 mm width×200 mm length×88 mm height.

FIG. 16 illustrates flow rate (L/min or LPM) vs shaft speed (Rev/min or RPM) of a pump contemplated herein comparing 4″ and 6″ Roller Pumps, two length of cams (8.4 and 14.3 cm). FIG. 16 illustrates testing with custom extruded silicone tubing, such as that shown in cross section in FIG. 11 and FIG. 12.

FIG. 17 illustrates a top-level assembly drawing of a pump device as contemplated herein. Table 1 describes the enumerated features of the pump device of FIG. 17.

FIG. 18 illustrates one embodiment of a pump as disclosed herein, opened (solid cam shown).

FIG. 19 illustrates one embodiment of a pump as disclosed herein, closed, with overall dimensions (excluding flat base portion and controller) of 101 mm width×223 mm length×64 mm height.

FIG. 20 illustrates one embodiment of an initial ball bearing cam concept using a flange 12 for interface to the motor. This is a flange-mounted version of the cam. The flange 12 is the round part on the end with mounting holes.

FIG. 21 illustrates one embodiment of a ball bearing cam concept assembly with pressed on gear and bolt on end cap. This ball bearing cam can provide forward flow. In this embodiment of the cam, a gear 16 is pressed onto a cap that is threaded onto the cam, eliminating the need for flange (FIG. 20) in this configuration.

FIG. 22 illustrates one embodiment of a pump as disclosed herein with overall dimensions (excluding flat base portion and controller) of 48 mm width×193 mm length×55 mm height.

FIG. 23 shows one embodiment of a solid cam.

FIG. 24 shows one embodiment of a pump as contemplated herein with IGUS linear tables for horizontal and vertical occlusion and motor mounted above the cam.

FIG. 25A shows one embodiment of a ball bearing cam (Hirsh Precison, Boulder, Colo.) on a 5-axis mill. PTFE ball bearings shown. FIG. 25B shows the machined part before it was annodized. The meansurements of the cams shown FIG. 25A and FIG. 25B are 26 mm long transition (taper) at inlet, 76 mm long fully occluded length and 26 mm long transition at outlet. Ball bearings have three degrees of freedom (x, y and z) and worked very well in reducing friction.

FIG. 26 shows exemplary results from flow testing in both ¼″ and ⅜″ circuit and through a 12 Fr cannula which increases resistance. These results used the cam lengths shown and described in FIG. 25A and FIG. 25B.

FIG. 27 shows an exemplary comparison of power requirements for different ball bearing materials (ceramic, ptfe and delrin). Ceramic ball bearings are optimal of the three materials tested.

FIG. 28 shows exemplary results from hemolysis testing performed on human blood. The cam pump was compared to a roller pump at a flow rate of 1 LPM. A control sample was kept at body temperature and also tested.

FIG. 29 shows the same hemolysis results that are shown above but normalized per impact (insult). Since the cam tested rotated at a higher speed to achieve an equivalent flow to the 4″ roller pump tested, the results were divided by the number of roller contacts or cam impacts. For the cam tested (76 mm full occlusion length), it rotated at 244 RPM to achieve 1 LPM of flow. The roller pump rotated at 64 RPM and makes 2 roller impacts per revolution.

FIG. 30 illustrates one embodiment of a portable pump assembly concept that can clamp anywhere on a tube to provide flow.

FIG. 31 shows one embodiment of a pump as contemplated herein with one occlusion adjustment for a more compact design.

FIG. 32 shows a profile view of a roller type cam embodiment of the current invention. Rollers may be used to reduce friction between the cam and tubing. Rollers may be preferred in some embodiments. It was decided that there would be some scraping of the rollers since they are limited to one degree of freedom (rotating along their center axis).

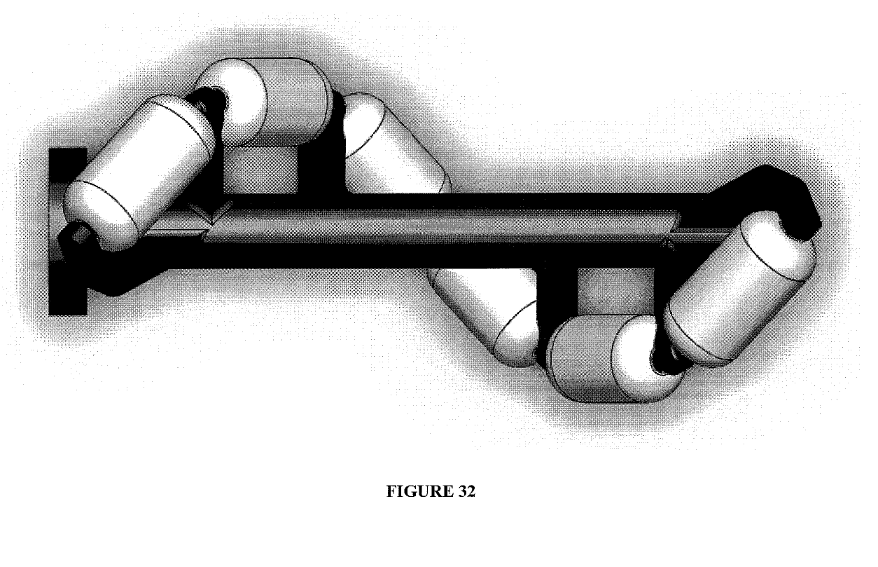

FIGS. 33A&B provide a comparison of pump technology utilized for heart lung machines. FIG. 33A depicts the commercially available heart lung machine from Terumo, also called the Advanced Perfusion System 1. FIG. 33B depicts pumps of the current invention being used in a heart lung machine capacity. The pumps are shown, mast mounted and placed very close to the patient bed. It is believed that the current invention design (FIG. 33B) makes tubing more efficient (less awkward) because the pumps allow for in-line flow. The roller pumps (FIG. 33A) require that the tubing be wrapped in a semi-circle in the pump raceway and require reliefs (partial loops) at the inlet/outlet to prevent tube kinking. This comparison indicates the economy of the design by reducing required tubing length.

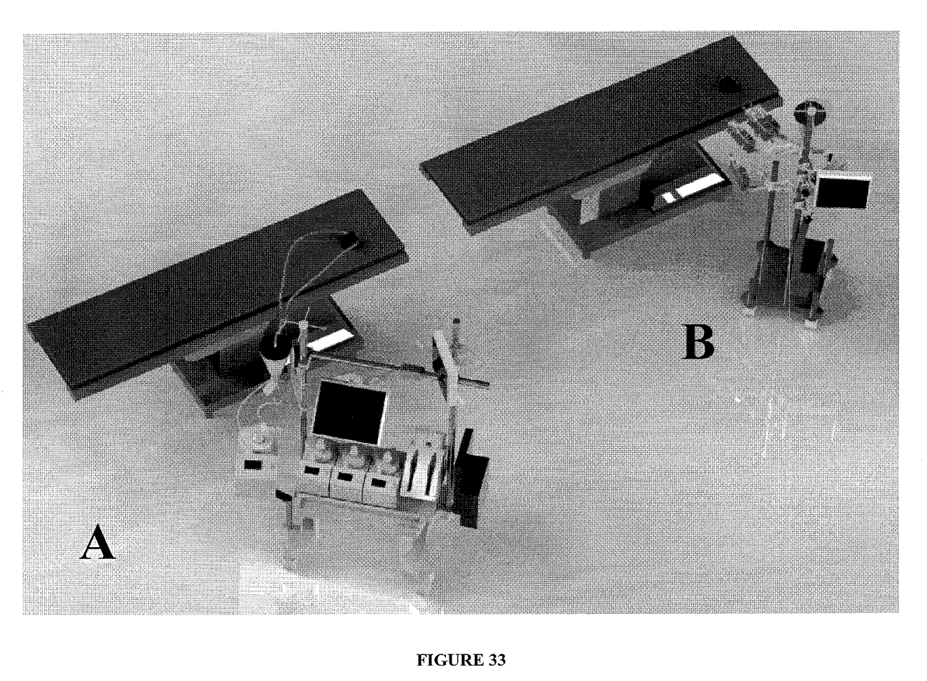

### DETAILED DESCRIPTION OF THE INVENTION

The present invention relates to pumps, particularly a compact, linear, positive displacement pump that can be used in several applications including medical and non-medical devices. In particular, the invention is related to linear, positive displacement compression blood pumps (either intracorporeal or extracorporeal) that provide systemic circulatory stability by maintaining a steady average blood pressure. In some embodiments, the invention relates to perfusion pumps, infusion pumps, pumps used for ECMO (extracorporeal membrane oxygenators) and bioreactor pumps.

Generally, blood pumps operate with different types of actuation and present challenges with respect to size, speed, and flow. For instance, and in various embodiments, roller pumps used in extracorporeal blood circuits are large and must be placed some distance from a patient, requiring tubing that spans from the patient to the pump and back to the patient. This length of tubing presents a number of risks to patients and recent trends in extracorporeal blood circuit design aim to mitigate these risks by reducing the amount of surface area, prime volume and banked blood products required to safely operate a heart/lung machine. These miniaturization efforts typically involve reducing tubing diameter and length and reducing the size of circuit devices.

Clinical specialists along with corporate engineers have made progress in miniaturizing extracorporeal circuits by reducing the size of circuit devices such as oxygenators and by reducing tubing diameter and length. These miniaturization efforts aim to reduce circuit prime volume and surface area. The amount of foreign surface area material presents a list of risks to patients such as acute activation of multiple coagulation and inflammatory factors. Specifically, these reactions are caused by blood contact with non-biological surfaces (tubing, pump, etc.). Additionally, the use of banked donor blood has inherent risks for patients such as blood borne pathogen exposure and increases the risk of infection. Minimizing the amount of prime volume in the circuit will reduce these risks to the patient, especially in neonates, where total blood volumes can range from 170 mL in premature babies to 300 mL in 3.5 kg babies. Typical neonatal extracorporeal circuits require 250-300 mL of prime volume which can be more than the patients circulating blood volume which requires that the extracorporeal circuit be primed with banked blood products. Current roller pumps are very bulky and the inlet is 360 Degrees from the outlet, making it very difficult to place the pump closer to the patient and efficiently reduce tubing length. These challenges put existing roller pumps at a disadvantage for use in a compact circuit. Centrifugal blood pumps are easier to incorporate into miniature circuit designs, however, the prime volumes of current centrifugal pump designs are too great to be of clinical value for neonatal extracorporeal circuits. Mast mounted roller pumps have recently become available in an effort to reduce tubing lengths of the circuit. However, these mast mounted roller pumps are still quite bulky and configuration of these pumps on the heart/lung machine is very congested and complicated which limits their clinical value. Miniaturized pumps for neonatal applications may include local assist for small (for example, but not limited to, 1-5 mm diameter) blood vessels (which may be either, or both, natural and artificial).

There remains a strong clinical need for a pump that functions with minimal risk to patients, in a form that is easily adaptable into a miniaturized circuit. Various embodiments of the invention described in this specification provide in-line (where the inlet is in-line to the outlet) flow with the flexibility to clamp onto tubing anywhere along an extracorporeal circuit. The device can be placed within close proximity to the patient and include the integration of the other main components in an extracorporeal circuit including the oxygenator, reservoir with an integrated heat exchanger.

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

Various embodiments described herein relate to devices and methods for pumping liquids or solids, for example, blood. For medical applications, the invention decreases risks to the patients by minimizing part contact with blood and can be used as an intracorporeal or extracorporeal pump. Embodiments of the invention provide pulsatile flow from a compact design without the need for artificial valves. To accomplish this, a uniquely shaped helical cam is utilized that is driven by a motor and used to compress an adjacent, flexible chamber in a peristaltic type manner providing unidirectional flow. In one embodiment, said cam varies in length. A longer cam creates more flow per revolution because it displaces more volume. The only non-biological item making contact with blood is the pumping chamber. In one embodiment, said pump is configured to perform as on-medical applications needing continuous supporting flow.

A benefit for extracorporeal applications is that the cam and pumping chamber require a small object volume, allowing the device to be placed in close proximity to the source (patient, fluid reservoir, etc.), thus reducing the length of the tubes providing flow to and from the pump. This reduction in tube length can also be beneficial to a patient because it reduces blood contact with non-biological materials (the tube) and can decrease patient morbidity.

Another feature of this design is the amount of pressure and flow control available by adjusting certain aspects of the cam. Testing has shown that these controls have a direct affect on flow rate, inlet/outlet pressure and pressure waveforms allowing for application specific fine-tuning.

### Pump Applications

The current invention pump may be used in various types of pump applications, examples of which are described below.

### Perfusion Pump

Perfusion pumps are used in open-heart surgery where the heart can be completely stopped. There are typically five pumps used in a perfusion system, the main being the arterial pump, which acts as the ventricles, supporting all cardiac output. The other pumps are the carioplegia pump, which pumps a solution into the coronary arteries to stop the heart. The other three pumps are called suckers, which basically suck blood back to the venous reservoir. The pump of the current invention can be used for any of these pumps used in a bypass system. In one embodiment, said pump is configured to perform as on-medical applications needing continuous supporting flow.

### ECMO Circuit

ECMO (extracorporeal membrane oxygenation) pumps are devices for long-term cardiac and/or pulmonary support. In medicine, such pumps may work to improve the circulation of a subject suffering from poor circulation from a variety of conditions.

### Ventricular Assist Devices

Embodiments of the invention provide flow from a compact design without the need for artificial valves. In some embodiments, the invention provides pulsatile flow. To accomplish this, a uniquely shaped helical cam is utilized that is driven by a motor and used to compress an adjacent, flexible chamber in a peristaltic type manner providing unidirectional flow. In one embodiment, said pump is configured to perform as on-medical applications needing continuous supporting flow. The only non-biological item making contact with blood is the pumping chamber. For instance, and in various embodiments, ventricular assist devices (VADs) operate at very high speeds. This may present risk with respect to hemolysis, thrombosis, and micro-embolism. Continuous flow Ventricular Assist Devices (VAD's) present risks to patients as they create flow by utilizing a rotor that is placed in direct contact with blood, rotating at speed of up to 10,000 RPM. These fast spinning rotors are used in both axial and centrifugal flow pumps and can cause hemolysis and thrombosis in patients. Existing pulsatile flow VAD's are bulky and require valves to control flow, which also presents thrombogenic risks to the patients. In addition, there is an increase in non-biological materials making direct contact with blood, which can increase patient morbidity. There remains a clear clinical need for a compact VAD that provides pulsatile flow without the need for artificial valves.

### Infusion Pump

An infusion pump infuses fluids, medication or nutrients into a patient's circulatory system. It is generally used intravenously, although subcutaneous, arterial and epidural infusions are occasionally used. Infusion pumps can administer fluids in ways that would be impractically expensive or unreliable if performed manually by nursing staff. For example, they can administer as little as 0.1 mL per hour injections (too small for a drip), injections every minute, injections with repeated boluses requested by the patient, up to maximum number per hour (e.g. in patient-controlled analgesia), or fluids whose volumes vary by the time of day. In one embodiment, said pump is configured to perform as on-medical applications needing continuous supporting flow.

Because infusion pump can also produce quite high but controlled pressures, infusion pump can inject controlled amounts of fluids subcutaneously (beneath the skin), or epidurally. The user interface of pumps usually requests details on the type of infusion from the technician or nurse that sets them up:

Continuous infusion usually consists of small pulses of infusion, usually between 500 nanoliters and 10 milliliters, depending on the pump's design, with the rate of these pulses depending on the programmed infusion speed.

Intermittent infusion has a “high” infusion rate, alternating with a low programmable infusion rate to keep the cannula open. The timings are programmable. This mode is often used to administer antibiotics, or other drugs that can irritate a blood vessel.

Patient-controlled is infusion on-demand, usually with a preprogrammed ceiling to avoid intoxication. The rate is controlled by a pressure pad or button that can be activated by the patient. It is the method of choice for patient-controlled analgesia (PCA), in which repeated small doses of opioid analgesics are delivered, with the device coded to stop administration before a dose that may cause hazardous respiratory depression is reached.

Total parenteral nutrition usually requires an infusion curve similar to normal mealtimes.

Some pumps offer modes in which the amounts can be scaled or controlled based on the time of day. This allows for circadian cycles which may be required for certain types of medication.

The pump of the current invention provides advantages in its use as an infusion pump by providing in-line flow in a compact design. It is believed that the tuneable aspects of the pumping system (cam and pumping chamber) will provide superior accuracy with delivery control.

### Bioreactor Pump

In living systems, such as cell culture systems, it is important to have a flow of medium thoughout the growing space to maintain proper health and optimal growth conditions. A bioreactor pump aids in this process. The pump of the current invention provides advantages in its use as a bioreactor pump by the economy of space needed in use (less tubing) and elegancy of design. In one embodiment, said pump is configured to perform continuous supporting flow. In one embodiment, said pump is configured to perform pulsatile supporting flow.

### Tubing

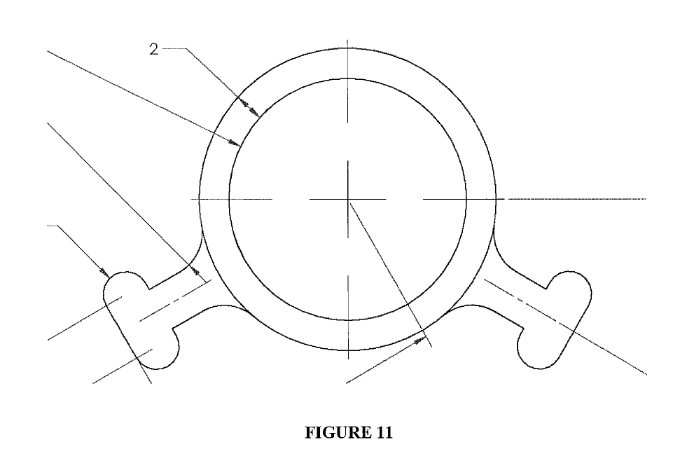

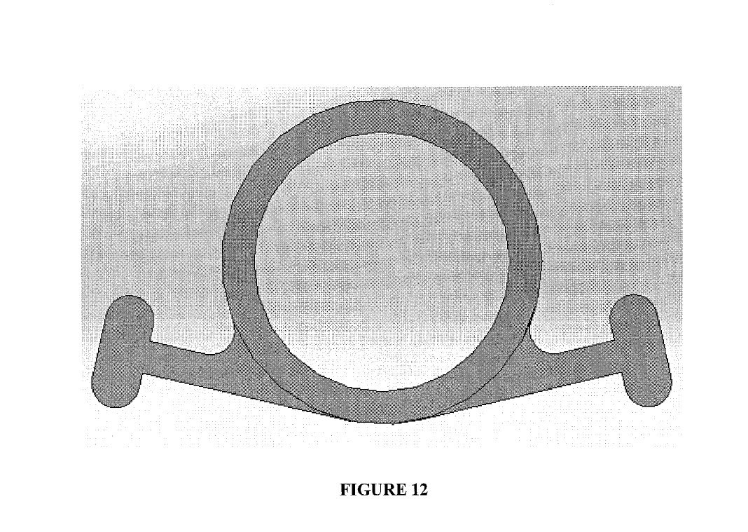

The present invention is not to be limited by the type of tubing used. In some embodiments, compressive materials such as PVC, silicone, rubber, etc. may be used for tubing. In some embodiments, the tube can have a standard circular cross section or a custom shape with features that allow for support along any edge (such as the custom tube shown in FIG. 11 and FIG. 12).

### System Design

The present invention is not limited to the particular pump designs described in the examples, but includes embodiments wherein the elements are varies. For example standard off-the-shelf linear stages were used to control occlusion (compression) of the tube by varying the distance between the cam and tube. The system of the present invention may emcompass any design that would allow for occlusion adjustment, both mechanical (such as manipulation of a turn of a dial or knob) or electro-mechanical (using motors/software).

In one embodiment, the invention further comprises a cap that screws onto the cam shaft. The cap may serve several purposes including, but not limited to: 1) the cap has a feature that retains the ball bearings and when removed, allows for loading of the ball bearings into a feature (track or friction reducing element track) in the cam, when installed, retains the ball bearings; 2) the cap has a feature that constrains the cam within a bushing that is supported by a bracket an the other end of the cam has the same feature that sits in a bushing, mounted in the same bracket; 3) the cap has constrains the cam assembly.

### Cad Models

Below is a brief description of each design phase with test results. In some embodiments, the cam may vary in length. A longer cam creates more flow per revolution because it displaces more volume.

### Phase 1: Proof of Concept

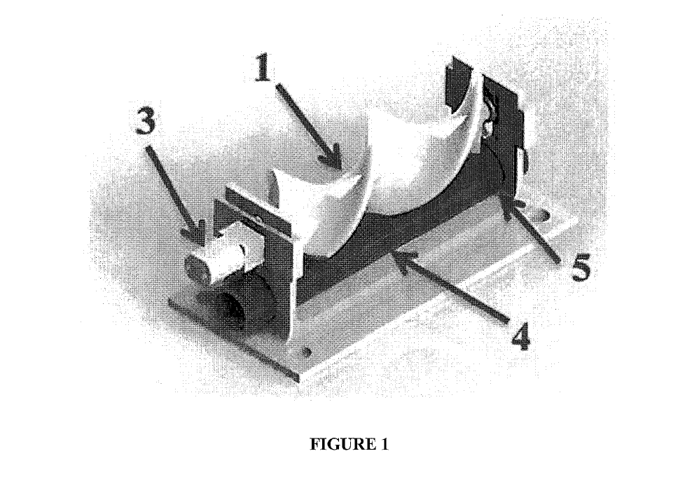

Test results suggested that this pumping method is successful for increasing flow rate. FIG. 1 illustrates a cam 1, a motor 3, a tube system 4, and a tube constraint 5.

### Phase 2: Alpha Prototype

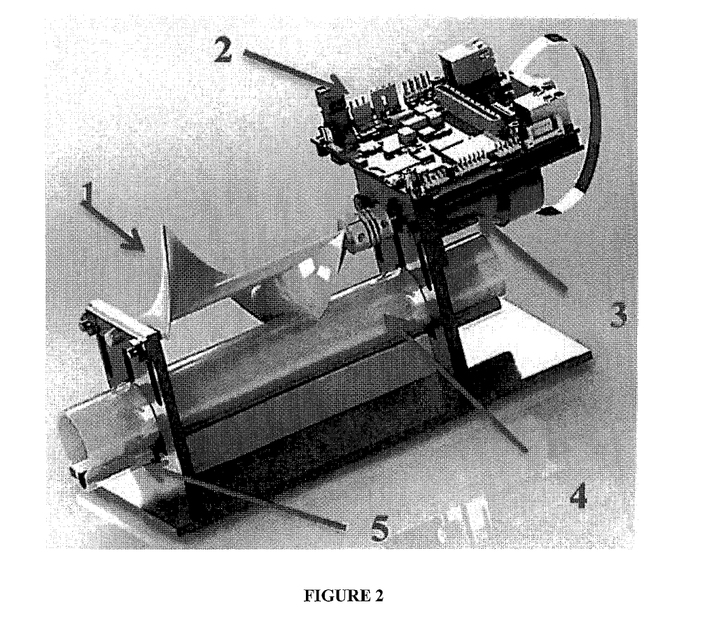

FIG. 2 illustrates a cam 1, a controller 2, a motor 3, a tube system 4, and a tube constraint 5. Test results confirmed that the design acts as a forward pump, providing a consistent 25% increase in flow rate. In addition, pressure data was collected at the inlet and outlet demonstrating how the pressure can be controlled with modifications in the cam shape in all three cam shapes tested.

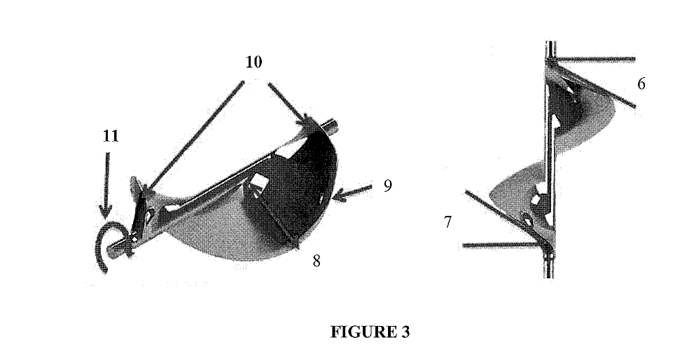

FIG. 3 illustrates cam control with an inlet angle 6, an outlet angle 7, a cam height 8, a cam profile edge 9, the number and degree of rotation of helix 10 (360 degree shown), and rotation 11 related to speed based on shaft speed of motor.

### VAD Clinical Application Example

In one embodiment, the present invention provides an external controlled compression pump for pediatric patients that have undergone a Fontan procedure who are experiencing failing hemodynamics, requiring a need for a heart transplant. In one embodiment, the compression pump provides pulsatile, circulatory support to the right side of the heart, which would restore circulation to a state that closely resembles dual ventricle circulation with minimal risk to a patient. In addition, the pump would need to decrease pressure at the inlet (systemic venous pressure) and increases pressure at the outlet (pulmonary arterial pressure), which is commonly referred to as the Fontan paradox. These patients usually require a modest boost in flow (˜20%), which poses a problem for continuous flow VAD's, as they cannot operate at such low speeds.

In one embodiment, an external controlled compression pump can be implanted in the area of the Total Cavo-Pulmonary Connection (TCPC) to augment the left ventricle for Fontan patients needing a BTT device while they await a heart transplant. An extracorporeal version of the pump can be placed within close proximity to the patient to provide low risk, left ventricle augmentation as well. There is a clear clinical need for this device, because there are currently no approved pulsatile flow Ventricular Assist Devices (VAD's) that have gained widespread pediatric use as a BTT device and none that have specifically targeted patients with a failing Fontan.

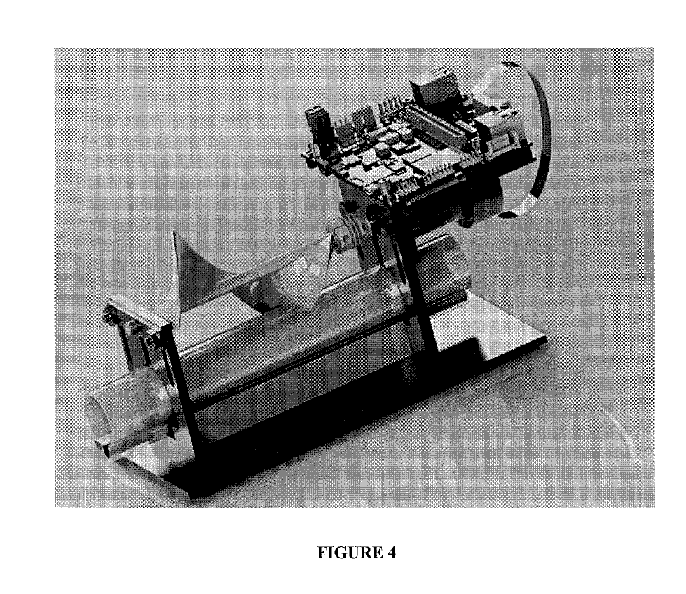

The data presented herein demonstrate that by externally compressing a tube with a cam that is attached to a motor shaft increases blood flow up to 25%. Pressure/time pulse data is also shown, as well as average inlet/outlet showing an average boost to arterial pressure of ˜22 mmHG, with a slight decrease in venous pressure at the inlet. FIG. 4 illustrates an exemplary embodiment of a pump having a tube with external cam compression.

### Design Basics

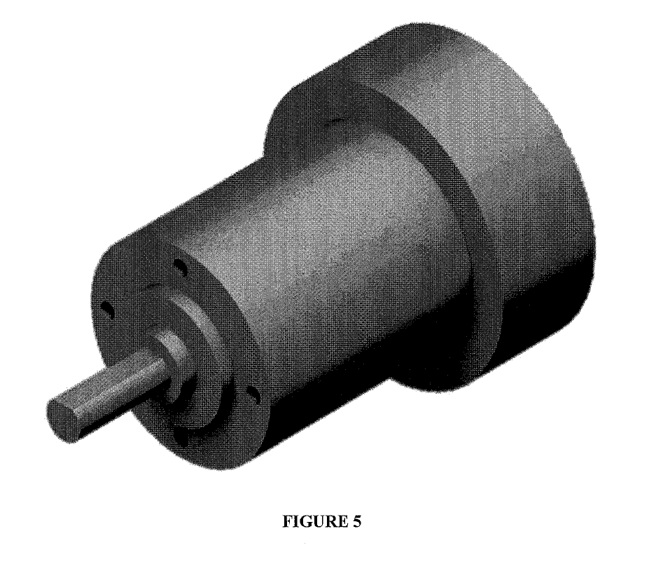

An external cam controlled compression pump design basically involves attaching a uniquely shaped helical cam to a motor shaft and as the shaft rotates, the cam follow and makes contact with a tube, squeezing it along its length in a peristaltic type manner. The motor used for the compression pump may be commerically available (e.g., Maxon Motors). FIG. 5 illustrates an exemplary embodiment of such a motor.

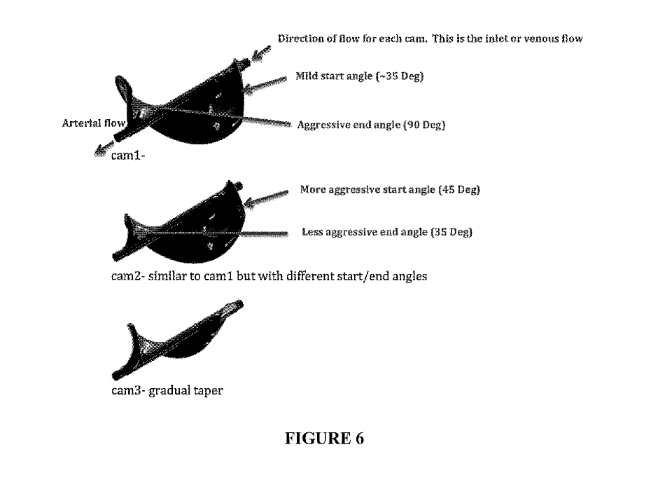

Three cams shapes were tested and all of the results shown below are from the same cam shape (cam 2), which demonstrated the largest increase in flow (˜25%). It could be beneficial to build a computer model to analyze flow with varying modifications to the cam. FIG. 6 illustrates the three cam designs. The cam can be made of plastic, metallic alloys or any material that is stiff enough to support the compressive loads generated. The center shaft and ridge may be one solid part or segmented into several parts that are linked. In some embodiments, the cam requires lubricant between the cam ridge and the tube.

### Data

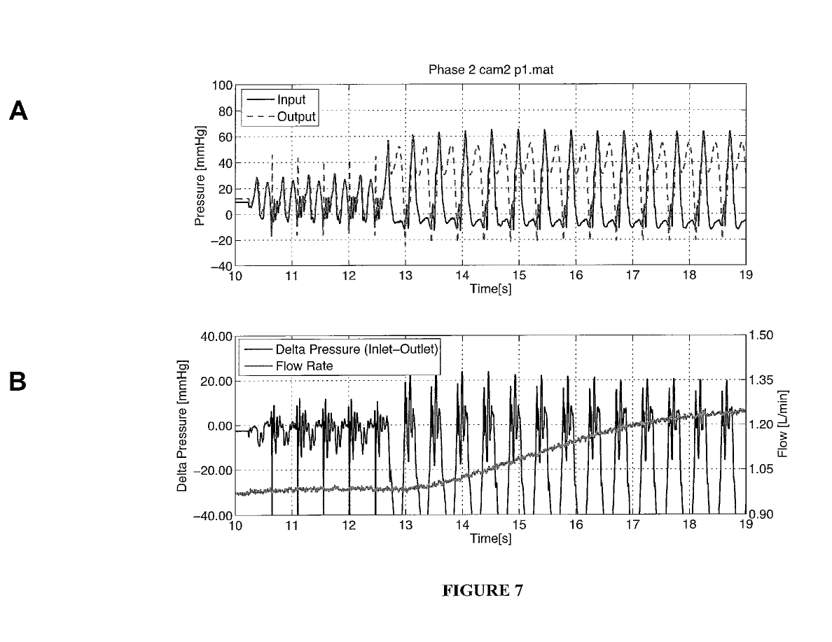

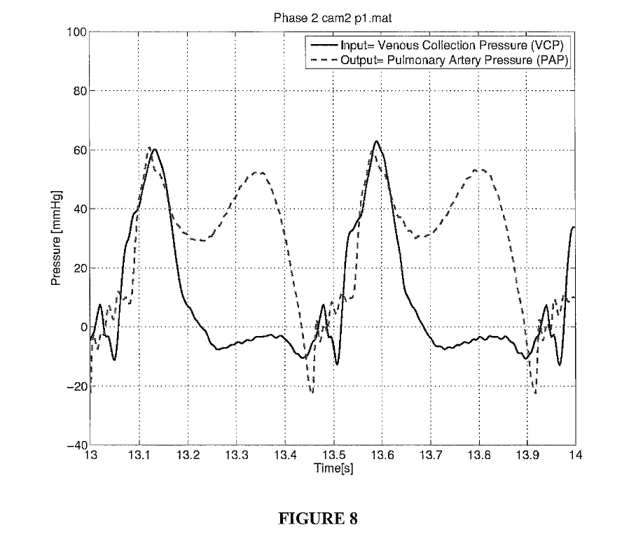

The graphs shown in FIGS. 7A&B show real time pressure/flow date plotted over a time interval for cam 2. The pump creates pulses (as shown in the pressure waveforms in FIGS. 7A&B and FIG. 8). The pump was run at a speed that would be continuous, but the pump could be made to be pulsatile with an appropriately sized cam and software that would control speed to create pulses.

FIG. 7A plots input pressure (upstream of venous collection pressure, VCP) and the output pressure (downstream or pulmonary artery pressure, PAP) plotted as a function of time with the pump on. The pump was turned on just after 10 s.

FIG. 7B plots the Delta of inlet outlet pressure and flow plotted with time, showing ˜25% in flow with pump turned on.

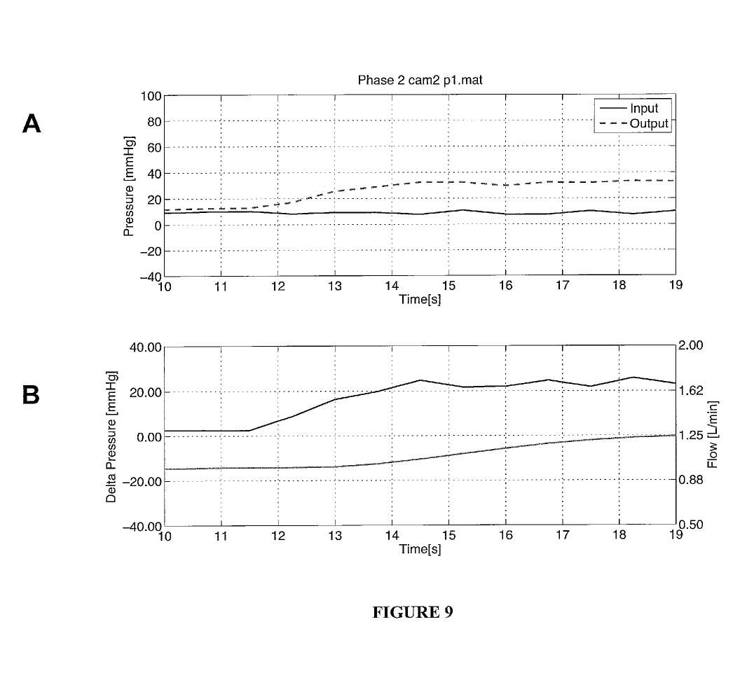

The plot shown in FIGS. 9A&B is a zoomed in view of the data shown in FIG. 7A, between 13 and 14 s, showing pressure pulses for both the inlet and outlet. Note that the pump speed is ˜125 RPM (2 pulses per second). The area under the curves is the flow energy of the system and the output area is greater than the input area, resulting in the net flow increase.

The average pressure levels present in the above data are in contrast to published papers/reports that have shown average flow increases over time. Although it is not necessary to understand the mechanism of an invention, it is believed that suction does not develop at the inlet because the flow is controlled by a baffle and is therefore relatively constant, thereby maintaining an average flow rate, even though there is an apparent decrease in pressure at the inlet.

The plot of FIG. 8 is a zoomed in view of the data shown in FIG. 7A, between 13 and 14 s, showing pressure pulses for both the inlet and outlet. Note that the pump speed is ˜125 RPM (2 pulses per second). The area under the curves is the flow energy of the system and the output area is greater than the input area, resulting in the net flow increase.

FIGS. 9A&B illustrates Flow rate (L/min or LPM) vs shaft speed (Rev/min or RPM): Data was collected using an external controlled compression pump comparing 4″ and 6″ Roller Pumps. The graphs show real time pressure/flow date plotted over a time interval for cam 2. FIG. 9A shows input pressure (upstream or venous collection pressure, VCP) and the output pressure (downstream or pulmonary artery pressure, PAP) plotted as a function of time with the pump on. The pump was turned on just after 10 s. The lower plot shows the delta of inlet and outlet pressure and flow plotted with time, showing ˜25% in flow with pump turned on.

### In Vitro Mock Circulation Set-Up

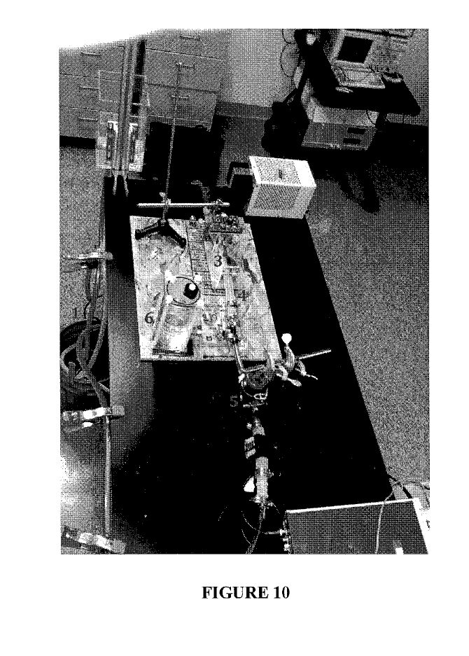

FIG. 10 shows an image of an in vitro mock circulation set-up, which includes the following components:

[1]—Single ventricle (left), continuous steady flow

[2]—Systemic venous collection (or feed from SVC and IVC). This is the pump inlet.

[3]—CPAD or cavopulmonary assist device

[4]—Pulmonary artery

[5]—Pulmonary vascular resistance

[6]—Pulmonary compliance

FIG. 11 illustrates a tube with attachment anchor portions.

Various embodiments provide different tube cam shapes. For example, there are several tube cam shape control features providing adjustability, including, but not limited to, cam lobe height, inlet/outlet angles, helical degree of rotation, etc. With this design, these control features can adjust the pressure/time/flow waveforms.

Some external cam control compression pump embodiments may comprise various additional features, including, but not limited to, a ridge along the edge of the cam to reduce friction, a sliding feature, a ball/roller bearing along the ridge, and/or strips of a low friction creating material. These additional features may be optimized to address biocompatibility and mounting in a patient's thoracic cavity.

### Electrical Engineering

Various embodiments contemplated herein may provide adjustments for both motor and/or control system design. In one embodiment, the pump is run at a certain speed and data collected. However, there may be control loops with flow/pressure feedback to control motor speed/position of cam. Motor sizing may be based on required draw for fluid pumping.

One advantage of the presently disclosed external cam controlled compression pump is the method by which fluid is moved through the pump. For example, to increase flow, the device converts rotational motion on an axis parallel to an axis though the center of the tube, to linear compression of an area of the tube in a repeatable manner. The linear speed of the compressed area is directly proportional to the rotational speed of the motor and is explained in further detail under the flow control section.

### Inlet/Outlet Angle:

The inlet and outlet angles of the cam can vary between greater than 0 Degree (Deg) and less than 90 Deg. Data was collected from two different cam embodiments (e.g., Cam 1 and Cam 2), with the following parameters:

Cam 1: start angle 35 Deg, end angle 90 Deg

Cam 2: start angle 45 Deg, end angle 35 Deg

### Cam Height:

The height of the cam (from the center of rotation) can vary indefinitely but must be tall enough to provide adequate compression of the tube. The most recent test data shows that the tube must be compressed a minimum of 90% of its height to provide flow.

### Cam Profile Curvature/Width

To reduce friction between the cam and the tube, a full radius was added to the outer edge. The width of the cams tested was 7 mm, but this can vary, depending on the size of the tube being compressed.

An alternative design can involve utilizing a series of rollers that are offset from the center of rotation and angled in a manner that follows a helical path.

This device can be connected to internal organs for increased local perfusion.

Intracorporeal Example:

Fontan palliation involves diverting all systemic venous returns (Superior and Inferior Vena Cava) directly to the pulmonary arteries. This is referred to as the Total CavoPulmonary Connection (TCPC) and is illustrated below.

Extracorporeal Example:

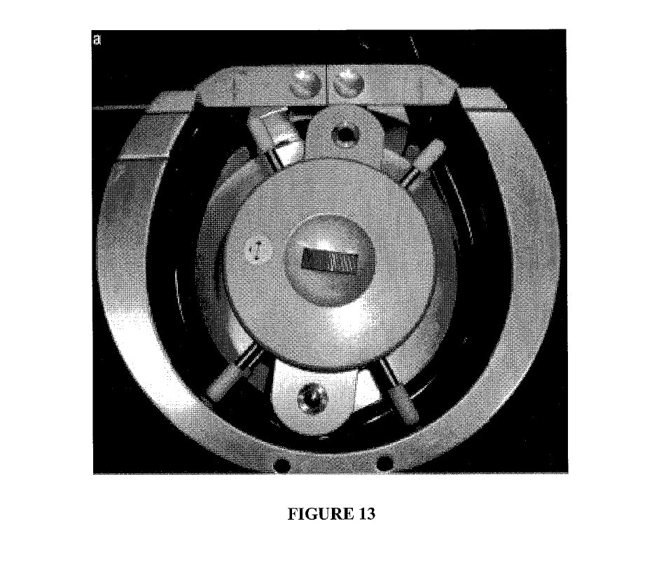

FIG. 13 shows a typical roller pump used in a cardiopulmonary bypass machine. These roller pumps are bulky and can be replaced with an extracorporeal version of this invention.

This device can also be used in bioreactors for growing new blood vessels and as a small, self-contained ECMO (Extra Corporeal Membrane Oxygenator) device.

Wiring for the controller and battery pack may extend outside of the body. The controller and battery pack may be contained in a pack and worn by a patient.

For failing Fontan patients, a modest boost in downstream pressure of 2-5 mmHg would greatly improve circulation. For other uses, the pressure can range to a maximum of ˜120 mmHG.

To control pressure, the inlet and outlet angles of the cam can be modified. The steeper the outlet angle, the higher the pressure at the outlet and the steeper the inlet angle, the higher the suction is at the inlet.

Low flow, ˜20% boost for failing Fontan patients. Flow is proportional to the motor shaft speed. Volumetric flow of blood can be converted into fluid velocity through the pump tubing by dividing by the cross sectional area of the tube. The edge of the cam making contact with the tube, acts as an external wiper that is moving at a linear speed proportional to the motor speed. To allow for forward flow, the speed of the motor (and consequent linear speed of the cam) must be traveling faster than the fluid traveling through the tube.

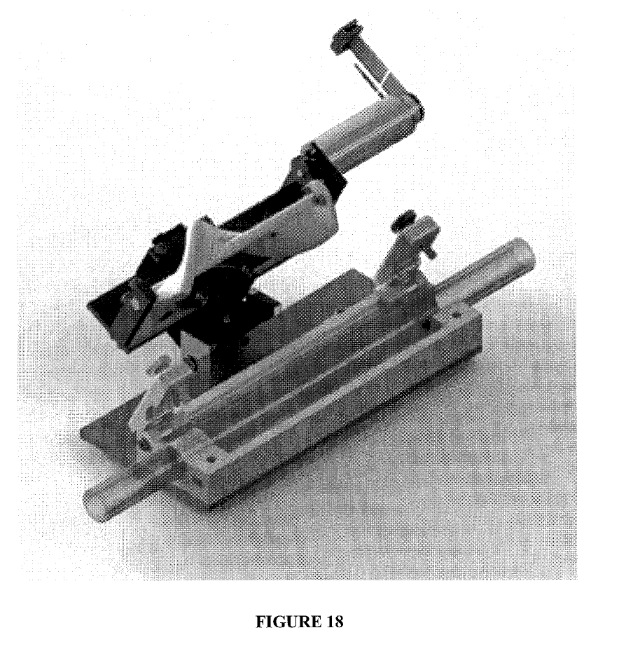

Materials may include, but are not limited to: Motor/Gearbox/Sensor/Cables/Controller from Maxon Motors. The hardware, which is all referenced and purchased from McMaster Carr. Bushing, purchased from IGUS. Additionally, the company (Surface Solutions) out of Chicago, coats samples of the cam and tube. The tubing for the Beta prototype sample may be purchased from Specialty Manufacturing, Inc. out of Saginaw, Mich. The tubing profile for this design (illustrated with standard, round tubing in FIG. 18 or with cusom tubing seen in cross section in FIG. 11 and FIG. 12 with a 16 mm ID custom profile) and may be extruded from a silicone material from Dow Corning called, Silastic BioMedical Grade ETR Elastomers (Q7-4750). This profile may change slightly but the general concept will remain consistent, that it will be a round or elliptical cross-section with “legs”, 1 on each side as the profile in FIG. 11 and FIG. 12 show. In preferred embodiments, standard PVC tubing is used in conjunction with the pump, particularly in bypass applications.

List of potential materials used may include, but are not limited to: Housing: Titanium, Stainless Steel. Tube: Silicone, Gore Tex, Natural Rubber. Cam: Stainless Steel, Titanium, Aluminum, Plastic. Bushing: Plastic. Support Structure: Plastic, Titanium. Coating for tube/cam: Slick Sil.

This pump may have other applications, which include either medical or non-medical: drug delivery, infusion pump systems, RVAD, LVAD, or BiVAD, vein dilation for AV fistulas, a bioreactor pump, and a self-contained extracorporeal membrane oxygenator. A bypass circuit with this pump may include additional features including an oxygenator, a heat exchanger, and a venous reservoir.

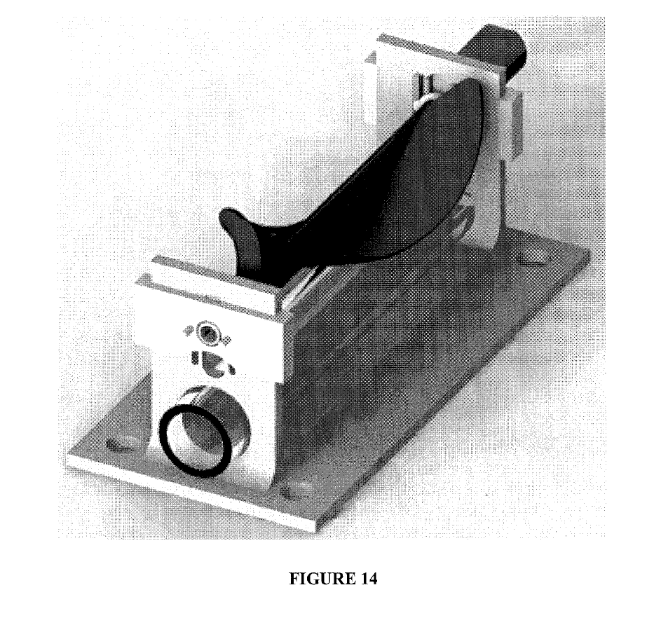

FIG. 14 illustrates a rendering of a proof of concept embodiment. The overall dimensions of this exemplary embodiment (excluding flat base portion) may be 30 mm width×135 mm length×47 mm height. The proof of concept was designed and built to demonstrate that forward flow can be achieved by externally compressing a tube with a helical cam shape. A test circuit includes a simple flow circuit that was configured, using a continuous flow pump. The baseline flow rate was set at 1 L/min and the pump was added to the circuit to augment flow.

Materials: Motor: Solarbotics p/n GM20 with the stall torque (6V) 37.70 in*oz (266 mN-m), the gear ratio is 100:1, and the unloaded RPM (6V) is 240. Controller: Solarbotics p/n 52231, 12 VDC, 2 A. Additional machined and off the shelf parts were used for construction of the prototype.

Results: A maximum of 6% increase in flow was achieved. The motor selected for this application did not provide adequate nominal torque for continuous testing. For a tubing constraint, a curved feature was added to the base to constrain the tube. Since there is a lateral load on the tubing as the cam rotates, the tube is forced out of position. A wall was added to the base to prevent this but it is not optimal for occlusion.

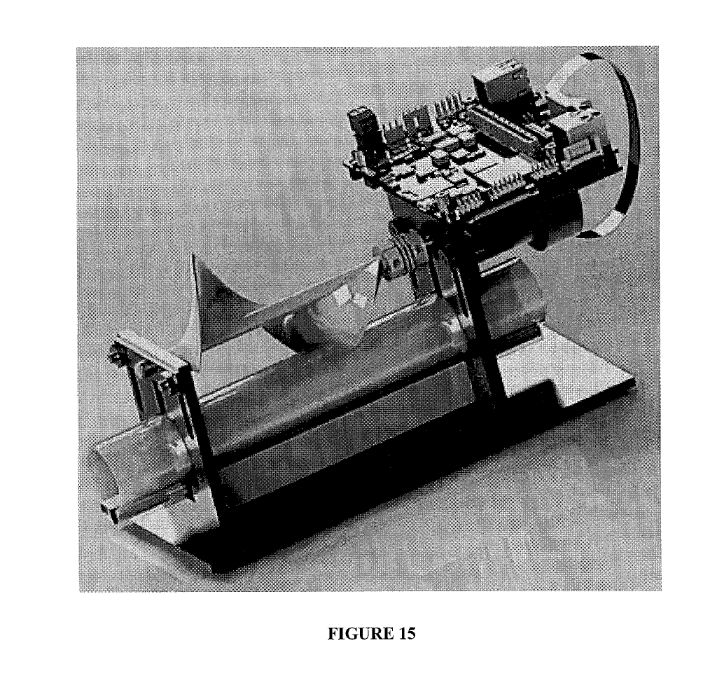

FIG. 15 illustrates a rendering of an alpha prototype. Overall dimensions (excluding flat base portion and controller) may be 35 mm width×200 mm length×88 mm height. The alpha prototype was designed to improve flow based on the limitations of the proof of concept. Key modifications made to this prototype include: full constraint of the tube along the bottom, and a motor capable of providing much more torque. A test circuit includes a simple flow circuit that was configured, using a continuous flow pump. The baseline flow rate was set at 1 L/min and the pump was added to the circuit to augment flow.

Materials: Motor: Maxon EC45 flat 30 Watt, brushless DC motor (part number 339282) with Nominal Voltage 36V; Max continuous Torque: 66.6 mN-m; Stall Torque: 337 m-Nm; and Nominal speed: 3,290 RPM. Gearbox: Maxon, GP 32C part number 166937 with a 28:1 gear ratio. The max speed after gear ratio is 118 RPM (3290/28). The shaft diameter is 3 mm. The controller is a Maxon part number 367661. There is a DEC 24/2 module with Eval board, part number 370652. Additional machined and off the shelf parts were used for construction of the prototype. Results included a maximum of 25% increase in flow. Pressure waveform and flow data was also collected.

Some potential limitations are discussed herein below. Although occlusion was improved by constraining the tube along the entire length of the race way, occlusion was not maximized. To maximize occlusion, a base that constrains the tube with geometry that matches the cam profile or a tube that is constrained in a manner that limits the lateral motion of the tube as it is compressed by the cam.

Furthermore, constraining the round tube by pinching it along the bottom, deformed the round tube into an elliptical shape which effects the interaction between the cam and tube. Lastly, excessive friction is generated between the tube and the cam.

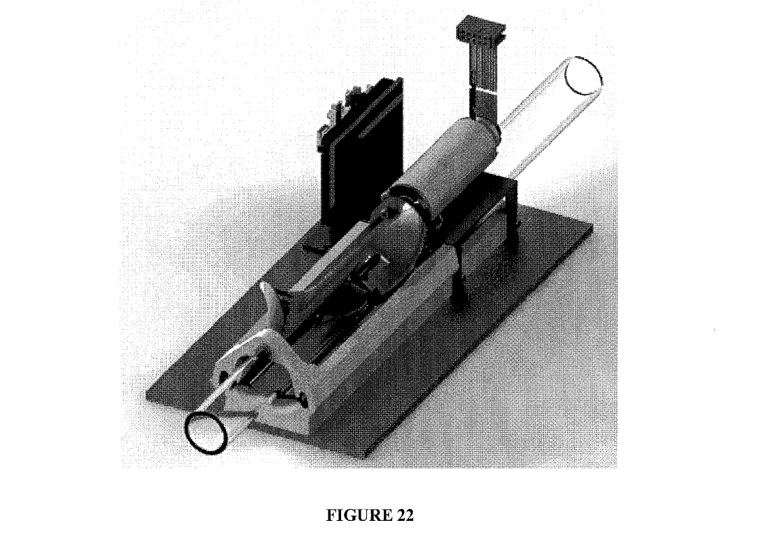

FIG. 22 illustrates a rendering of a beta prototype. Overall dimensions (excluding flat base portion and controller) may be 48 mm width×193 mm length×55 mm height. The beta prototype was designed to improve flow based on the limitations of the alpha prototype. Key modifications made to this prototype included: creating a custom tube extrusion with two raised sections (legs) that slide into the base. These features constrain the tube along the lower part of the tube and allow compliance of the lower wall. The silicone tube was coated with a friction reducing material called SlickSil provided by Surface Solutions which should reduce friction by 25%. A custom base was created to constrain the tube using the protruding leg features. A custom motor mount was created to mount the motor and attach it to the base. The cam was created with a flange 12 to attach to a custom flange 13 that attached to the motor shaft. A smaller motor was selected based on torque measurements on the alpha prototype.

A test circuit includes a simple flow circuit that was configured using a venous reservoir. The pump was not set up to augment flow as in previous testing, but rather to provide flow to/from the reservoir.

Materials: Motor: Maxon DCX22S, 24 W brushless DC motor, Nominal Voltage 24V; Max continuous Torque: 15.3 mN-m; Stall Torque: 120 mN-m; and Nominal speed: 10,800 RPM. Gearbox: Maxon planetary gearhead GPX22 A p/n 166937 with a 35:1 gear ratio. Max speed after gear ratio is 309 RPM (10800/35). Shaft diameter: 4 mm (with 2 flats). Configuration: Maxon motor and gearbox (items 1 and 2) part number B715857024A8 with a max diameter: 22 mm, length 67.3 mm (includes encoder, excludes shaft); no load speed: 354 rev/min; nominal torque: 401.4 mN-m; and nominal current: 0.869 A. Controller: Maxon EPOS 24/2 positioning control unit, p/n 390438 with an operating voltage: 9-24 VDC; max current: 4 A; motor eventually changed to: Maxon DCX22L, 19.8 W brushless DC motor, part number B72D08514685 having a nominal Voltage 24V; max continuous Torque: 29.2 mN-m; stall Torque: 150 mN-m; nominal speed: 5,060 RPM; gearbox: Maxon planetary gearhead GPX22 A part number 166937; 28:1 gear ratio; and max speed after gear ratio is 181 RPM (10800/35). Shaft diameter: 4 mm (with 2 flats).

Configuration: Maxon motor and gearbox (items 5 and 6) p/n B72D08514685 with a max diameter: 22 mm, length 80.3 mm (includes encoder, excludes shaft); no load speed: 145 rev/min; and nominal torque: 662.3 mN-m; nominal current: 0.656 A. Additional machined, off the shelf parts and rapid prototype parts were used for construction of the prototype. Results included generation of 4.23 L/Umin of flow with an 8.4 cm cam and 5.00 Umin of flow with a 14.4 cm cam. Both flow values are based on the maximum shaft speed that could be generated with this motor/gearbox configuration, which was 271 RPM. Both cam lengths mentioned describe the effective length of the cam which is the length of cam that fully occludes the tube, excluding the length of the inlet/outlet transitions. The tubing was run for 3 hours continuous, without rupturing the tube.

Some potential limitations are discussed herein below. Motor interferes with tube at pump inlet. Need offset (gearing system) to avoid this. Aiming to use a standard (straight) PVC tube for the next prototype. Occlusion adjustment is needed. Current roller pumps have this feature.

Lubrication needed periodically to reduce friction between the solid cam and silicone tubing to reduce friction. Durability is a concern. The flange 12 to attach the cam to the shaft uses three bolts and requires a large diameter for mounting. Minimizing this diameter in a future prototype will be beneficial. The A shape feature at the end opposing the motor, used to constrain the opposite end of the cam interferes with the tube towards the end of compression. It is believed that this contact creates noise in the pressure vs flow plot.

FIG. 18 illustrates a rendering of a beta 2 prototype, opened.

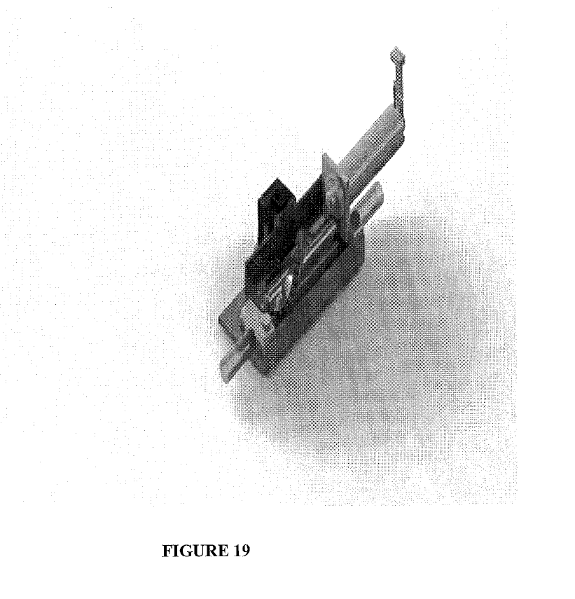

FIG. 19 illustrates a rendering of beta 2 prototype, closed. The overall dimensions (excluding flat base portion and controller) may be 101 mm width×223 mm length×64 mm height.

The beta 2 prototype was designed to eliminate motor interference with tube at pump inlet. The beta 2 prototype mounted the cam concentric to the motor shaft and the diameter of the motor interfered with the tubing slightly, which runs directly underneath the motor as shown in FIG. 19. The motor was mounted to a bracket that compressed the tubing slightly at the inlet.

This eliminates the need for a custom tube, allowing standard tubes typically used in a cardiopulmonary bypass circuit. This design added a capturing feature into the base to prevent the tubing from rotating past a certain point as the cam rotates. This design added a feature at the inlet and outlet to provide axial constraint of the tubing. This design adds a feature for occlusion adjustment. This was achieved by mounting the cam and motor to a bracket that attaches to a linear stages. The linear stage provides a maximum of 6 mm of travel in the z axis (up/down), allowing the amount the cam compresses the tube to be controlled. An offset gearing system was added to this prototype to eliminate this interference.

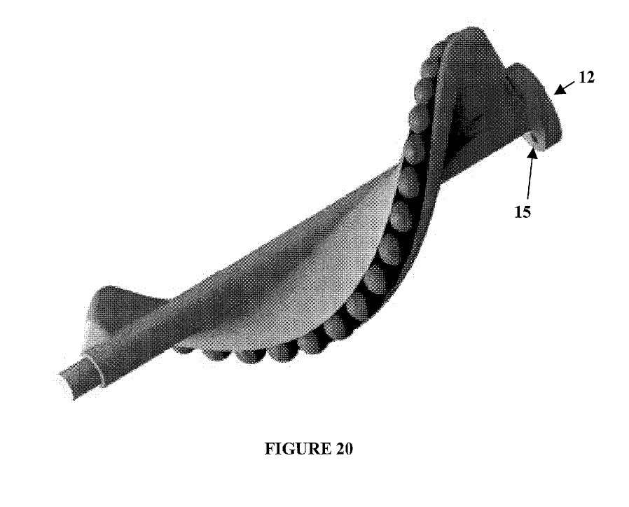

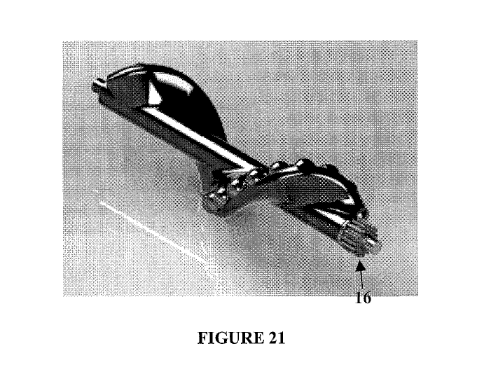

The cam can be made of plastic, metallic alloys or any material that is stiff enough to support the compressive loads generated. The center shaft and ridge may be one solid part or segmented into several parts that are linked. Initially, we started with a solid cam but kept having to add a lubricant between the cam and tube. To reduce friction between the cam and tubing, one configuration may align the outer ridge of the cam with ball bearings 14. Ball bearings have three degrees of freedom (x, y and z) and reduce friction. A couple renderings of concepts are shown in FIG. 19, FIG. 20, and FIG. 21. A gear (in the 2-gear system mentioned above) may be pressed onto the cam shaft to reduce the diameter required of the flange system. A cam with ball bearings may significantly improve pump function. FIG. 20 shows a flange-mounted version of the cam. The flange 12 is the round part on the end with mounting holes 15 in FIG. 20. FIG. 21 shows a system with a cam with a gear 16 is pressed onto a cap that is threaded onto the cam, eliminating the need for flange

The bracket system mentioned above eliminates the A shape cam constraint used in the beta prototype.

For a test circuit a simple flow circuit was configured, using a venous reservoir provided by the perfusion group. The pump was not set up to augment flow as in previous testing, but rather to provide flow to/from the reservoir.

Materials included motor. Maxon DCX32L, 105 W brushless DC motor, nominal Voltage 24V; max continuous Torque: 112 mN-m; stall Torque: 1,980 mN-m; nominal speed: 7,700 RPM; gearbox: Maxon planetary gearhead GPX32 A with a 35:1 gear ratio; shaft diameter: 8 mm (with single flat); configuration: Maxon motor and gearbox (items 1 and 2) part number B72E3A119583; max diameter: 32 mm, length 116.8 mm (includes encoder, excludes shaft); max load speed: 220 rev/min; nominal torque: 3,136 mN-m; and nominal current: 3.82 A.

Additional machined, off the shelf parts and rapid prototype parts were used for construction of the prototype. The prototype was built and flow has been achieved.

A ball bearing cam design, may greatly reduce friction between the tube and cam, allowing a smaller motor to be used (e.g., less friction to overcome) and this configuration will prolong the life of the tube.

FIG. 20 illustrates a rendering of an initial ball bearing cam embodiment using a flange 12 for interface to the motor.

FIG. 21 illustrates a rendering of a ball bearing cam embodiment assembly with pressed on gear and bolt on end cap.

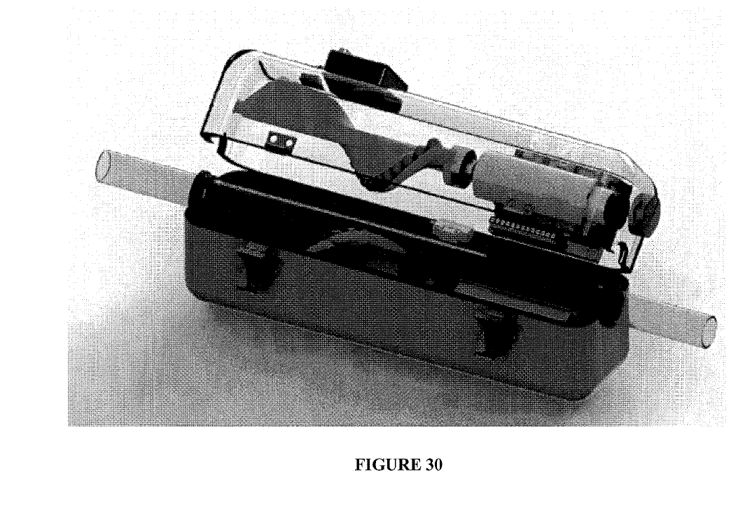

FIG. 30 illustrates a rendering of a clamp on a pump assembly.

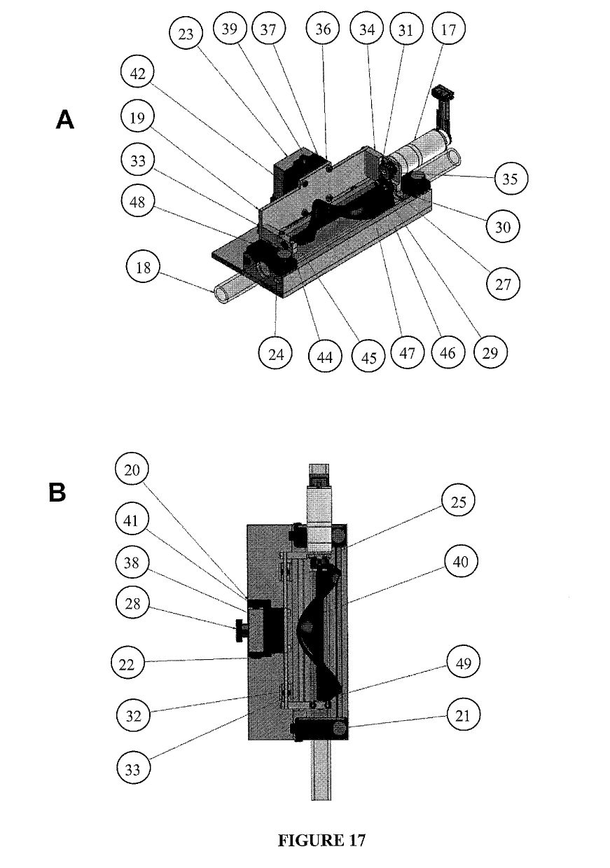

FIG. 17 illustrates a top level assembly drawing of a beta 2 pump device. Table 1 describes the features of the pump device of FIG. 17.

TABLE 1
Features of the pump device of FIG. 17.

| | | | Closed/Ball |
|---|---|---|---|
| ITEM | | | BearingT |
| NO. | PART NUMBER | DESCRIPTION | Cam/QTY |
| 17 | maxon_PN_B715857024A8-3 double flat | DCX22L with GPX22 28:1 | 1 |
| | shaft | gearhead/ENX16 easy sensor | 1 |
| 18 | 0.625 in 00 0.5 in ID cole parmer silicone | | 1 |
| | tube | | |
| 19 | cam and motor mount bracket | machined steel | 1 |
| 20 | pivoting base | machined aluminum | 1 |
| 21 | Misumi knob M4 nkosc4_16_14_2_03 | nkosc4-16-14 $6.40 | 2 |
| 22 | Misumi pivot pin c1bk5_45_2_03 | clbk5-45 $8.27 | 1 |
| 23 | pivoting base vert mount | machined delrin | 1 |
| 24 | threaded insert for clamp | machined aluminum, M4 | 2 |
| 25 | misumi dowel pin 1, 5 mm × 8 mm | MSC1, 5-8 $.,03 | 1 |
| 26 | Misumi 4 mm pin M3 thread | CLBK4-20 $8.10 | 2 |
| 27 | Misumi M3 threaded shaft bearing | NTSBG7-4 (W683ZZ 7 mm | 1 |
| | | bearing) $6.61 | |
| 28 | misumi knurled knob for pivot lock, M6 | NKOSC6-16-20 $9.53 | 1 |
| 29 | B18.3.1M-2.5 × 0.45 × 8 Hex SHCS- | | 3 |
| | 8NHX | | |
| 30 | misumi gear, without hub | GEFHBBO,5-30-4-4-WO (D = | 1 |
| | | 16, d = 15 $25.40 | |
| 31 | B18.3.1M-3 × 0.5 × 6 Hex SHCS--6NH | | 12 |
| 32 | HX-SHCS 0.164-36 × 0.625 × 0.625-N | | 2 |
| 33 | MSHXNUT 0.164-32-S-N | | 2 |
| 34 | motor mount bracket | machined steel | 1 |
| 35 | misumi gear, with hub | GEFHBB 0.5-30-4-4-W5-H6 | 1 |
| | | (D = 16, d = 15), shape B $25.30 | |
| 36 | misumi linear stage front | xkema40 $100.62 total | 1 |
| 37 | misumi linear stage back | xkema40 $100.62 total | 1 |
| 38 | B18.3.1M-4 × 0.7 × 12 Hex SHCS- | | 4 |
| | 12NHX | | |
| 39 | misumi linear stage top | xkema40 $100.62 total | 1 |
| 40 | 120 mm CAM | Polyjet | 1 |
| 41 | B18.3.1M-4 × 0.7 × 8 Hex SHCS--8NHX | | 4 |
| 42 | pointing plate | misumi pps 12 $2.47 | 1 |
| 43 | cam mount lower | | 1 |
| 44 | cam mount upper | | 1 |
| 45 | B18.3.1M-3 × 0.5 × 10 Hex SHCS- | | 2 |
| | 10NHX | | |
| 46 | Base 3 | machined AI 3/16 in plate | 1 |
| 47 | base with clamp 2 | SLA 0.5 in × 0.6875 in (0.094 in | 1 |
| | | wall) tubing | |
| 48 | tube clamp | | 2 |
| 49 | JFM_0608_06_1 | IGUS bushing | 1 |

In another embodiment, multiple tubes together with one cam may be configured to operate with one another. The tubes may be constrained around the perimeter of the cam, parallel to the cam rotational axis, with a case to properly constrain the multiple tubes.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

### EXAMPLES

The following examples are provided in order to demonstrate and further illustrate certain preferred embodiments and aspects of the present invention and are not to be construed as limiting the scope thereof.

### Example 1

### External Cam Compression Pump

### Objectives:

To create an external controlled compression pump for CPB (cardio pulmonary bypass) to be compared against extracorporeal roller blood pumps currently used in the clinical setting. The pump generates flow by utilizing a tapered helical shaped ridge that compresses tubing placed directly underneath, generating forward flow.

The CAM is expected to provide the following improvements:
1. Minimize length of tubing (by 15′-20′) used in cardio pulmonary bypass (CPB)
   a. Decreases CPB circuit surface area
   b. Decreases CPB circuit prime volume
2. Lower or equal hemolytic effect (as compared to a roller pump)
3. Clamp on pump over tubing, adaptable to any size of tubing, allowing for pediatric and adult use
4. In line flow vs horseshoe configuration in traditional roller pumps
5. The study of the flow characteristics may provide other physiological advantages to be determined.
6. Possible future uses:
   a. Portable IV/infusion pump (miniature size)
   b. Portable ECMO (Extracorporeal Membrane Oxygenator) unit
   c. Bioreactor (uses for in vitro tissue growth)

### Materials and Methods:

### Prototype Build and Test: Proof of Concept

The proof of concept was designed and built to demonstrate that forward flow can be achieved by externally compressing a tube with a helical cam shape (see FIG. 14). To generate flow, in a compact design, a shape was created that rotates with a motor and compresses the tube along its length to generate flow. To accomplish this, a ridge was created that follows a helical pattern and twists along a center support in the shape of a cylinder to give rigidity. The helical ridge is tapered on each end to provide a transition when initially compressing the tube and at the end of one full revolution, where the cam transitions off the tube to repeat the occlusion cycle.

### Test Circuit:

A simple flow circuit was configured, using a continuous flow pump. The baseline flow rate was set at 1 L/min and the pump was added to the circuit to augment flow.

### Materials:

1. Motor: Solarbotics p/n GM20
   a. stall torque (6V) 37.70 in*oz (266 mN-m)
   b. gear ratio is 100:1
   c. unloaded RPM (6V) is 240
2. Controller: Solarbotics p/n 52231, 12 VDC, 2 A
3. Additional machined and off the shelf parts were used for construction of the prototype

### Results:

A maximum of 6% increase in flow was achieved

### Proof of Concept Limitations:

1. The motor selected for this application did not provide adequate nominal torque for continuous testing.
2. Tubing constraint. A curved feature was added to the base to constrain the tube. Since there is a lateral load on the tubing as the cam rotates, the tube is forced out of position. A wall was added to the base to prevent this but it is not optimal for occlusion.

### Example 2

### Prototype Build and Test: Alpha Prototype

Description. The Alpha prototype was designed to improve flow based on the limitations (see FIG. 2) of the proof of concept. Key modifications made to this prototype include:
1. Full constraint of the tube along the bottom
2. Motor capable of providing much more torque

### Test Circuit:

A simple flow circuit was configured, using a continuous flow pump. The baseline flow rate was set at 1 L/min and the pump was added to the circuit to augment flow.

### Materials:

1. Motor: Maxon EC45 flat 30 Watt, brushless DC motor. p/n 339282.
   a. Nominal Voltage 36V
   b. Max continuous Torque: 66.6 mN-m
   c. Stall Torque: 337 m-Nm
   d. Nominal speed: 3,290 RPM
2. Gearbox: Maxon, GP 32C p/n 166937
   a. 28:1 gear ratio. Max speed after gear ratio is 118 RPM (3290/28).
   b. Shaft diameter: 3 mm
3. Controller: Maxon p/n 367661
   a. DEC 24/2 module with Eval board, p/n 370652
4. Additional machined and off the shelf parts were used for construction of the prototype.

### Results:

A maximum of 25% increase in flow was achieved. Pressure waveform and flow data was also collected, see below and FIGS. 7A&B)

### Pressure Waveform and Flow Data: Alpha Prototype.

The graphs shown in FIGS. 7A&B show real time pressure/flow date plotted over a time interval for cam 2 of the Alpha prototype. FIG. 7A shows input pressure (upstream or venous collection pressure, VCP) and the output pressure (downstream or pulmonary artery pressure, PAP) plotted as a function of time with the pump on. The pump was turned on just after 10 s. FIG. 7B shows the delta of inlet and outlet pressure and flow plotted with time, showing ˜25% in flow with pump turned on.

The plot shown in FIGS. 9A&B is a zoomed in view of the data shown in FIG. 7A, between 13 and 14 s, showing pressure pulses for both the inlet and outlet. Note that the pump speed is ˜125 RPM (2 pulses per second). The area under the curves is the flow energy of the system and the output area is greater than the input area, resulting in the net flow increase.

The plot of FIG. 8 is a zoomed in view of the data shown in FIG. 7A, between 13 and 14 s, showing pressure pulses for both the inlet and outlet. Note that the pump speed is ˜125 RPM (2 pulses per second). The area under the curves is the flow energy of the system and the output area is greater than the input area, resulting in the net flow increase.

Since most of the data reviewed in published papers/reports have shown average flow increases over time, average pressures were observed in this plot. Note: due to the current test set-up, it may be that the suction can be shown at the inlet with an average because the flow is controlled by a baffle and constant. However, it does appear that there is a slight decrease in pressure at the inlet.

### Alpha Limitations:

1. Although occlusion was improved by constraining the tube along the entire length of the race way, occlusion was not maximized. To maximize occlusion, a base that constrains the tube with geometry that matches the cam profile or a tube that is constrained in a manner that limits the lateral motion of the tube as it is compressed by the cam.
2. Constraining the round tube by pinching it along the bottom, deformed the round tube into an elliptical shape which effects the interaction between the cam and tube.
3. Excessive friction is generated between the tube and the cam.

### Example 3

### Prototype Build and Test: Beta #1 Prototype

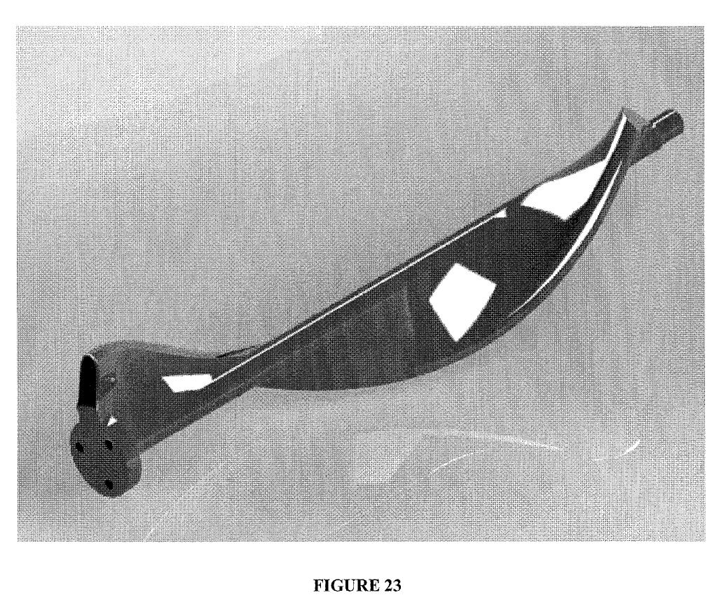

Description. The Beta prototype was designed to improve flow based on the limitations of the Alpha prototype. FIG. 22 shows the rendering of Beta #1 Prototype. Overall dimensions (excluding flat base portion and controller) 48 mm Width×193 mm Length×55 mm Height and FIG. 23 shows a rendering of the solid cam. Key modifications made to this prototype include:
1. Creating a custom tube extrusion with two raised sections (legs) that slide into the base. These features constrain the tube along the lower part of the tube and allows compliance of the lower wall, see FIG. 11 and FIG. 12.
2. The silicone tube was coated with a friction reducing material called SlickSil provided by Surface Solutions which should reduce friction by 25%.
3. A custom base was created to constrain the tube using the protruding leg features.
4. A custom motor mount was created to mount the motor and attach it to the base.
5. The cam was created with a flange 12 to attach to a custom flange 13 that attached to the motor shaft.
6. A smaller motor was selected based on torque measurements on the Alpha prototype.

### Test Circuit:

A simple flow circuit was configured, using a venous reservoir provided by the perfusion group. The pump was not set up to augment flow as in previous testing, but rather to provide flow to/from the reservoir.

### Materials:

1. Motor: Maxon DCX22S, 24 W brushless DC motor
   a. Nominal Voltage 24V
   b. Max continuous Torque: 15.3 mN-m
   c. Stall Torque: 120 mN-m
   d. Nominal speed: 10,800 RPM
2. Gearbox: Maxon planetary gearhead GPX22 A p/n 166937
   a. 35:1 gear ratio. Max speed after gear ratio is 309 RPM (10800/35).
   b. Shaft diameter: 4 mm (with 2 flats)
3. Configuration: Maxon motor and gearbox (items 1 and 2) p/n B715857024A8
   a. Max diameter: 22 mm, length 67.3 mm (includes encoder, excludes shaft)
   b. No load speed: 354 rev/min
   c. Nominal torque: 401.4 mN-m
   d. Nominal current: 0.869 A
4. Controller: Maxon EPOS 24/2 positioning control unit, p/n 390438
   a. Operating voltage: 9-24 VDC
   b. Max current: 4 A
5. Motor eventually changed to: Maxon DCX22L, 19.8 W brushless DC motor, p/n B72D08514685
   a. Nominal Voltage 24V
   b. Max continuous Torque: 29.2 mN-m
   c. Stall Torque: 150 mN-m
   d. Nominal speed: 5,060 RPM
6. Gearbox: Maxon planetary gearhead GPX22 A p/n 166937
   a. 28:1 gear ratio. Max speed after gear ratio is 181 RPM (10800/35).
   b. Shaft diameter: 4 mm (with 2 flats)
7. Configuration: Maxon motor and gearbox (items 5 and 6) p/n B72D08514685
   a. Max diameter: 22 mm, length 80.3 mm (includes encoder, excludes shaft)
   b. No load speed: 145 rev/min
   c. Nominal torque: 662.3 mN-m
   d. Nominal current: 0.656 A
8. Additional machined, off the shelf parts and rapid prototype parts were used for construction of the prototype

### Results:

Generated 4.23 L/min of flow with an 8.4 cm cam and 5.00 L/min of flow with a 14.4 cm cam. Both flow values are based on the maximum shaft speed that could be generated with this motor/gearbox configuration which was 271 RPM. Both cam lengths mentioned describe the effective length of the cam which is the length of cam that fully occludes the tube, excluding the length of the inlet/outlet transitions. The pump was run for 3 hours continuous, without rupturing the tube.

### Flow Results, Beta #1 Prototype

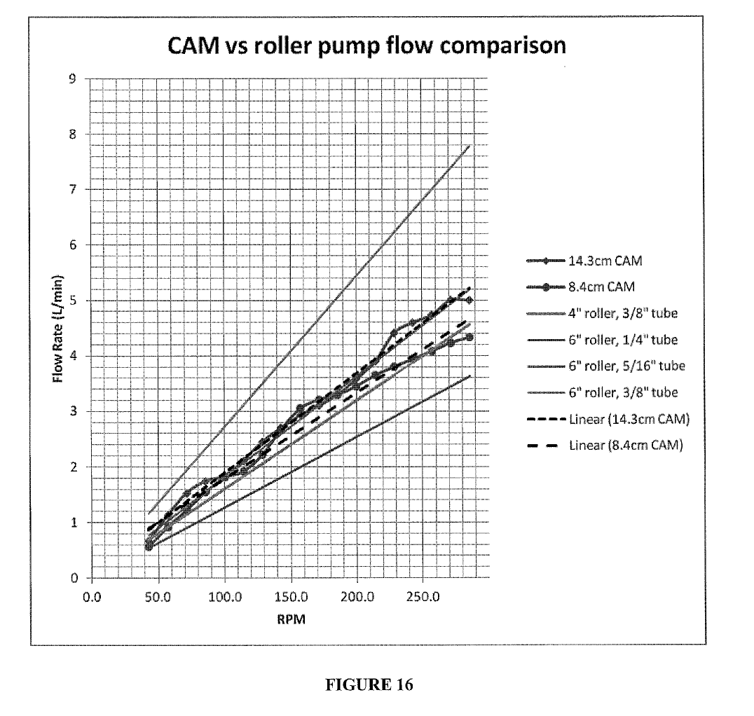

FIG. 16 shows the Beta #1 prototype flow results for 2 length of cams (8.4 and 14.3 cm) compared to a 4″ and 6″ roller pump.

### Beta Limitations:

1. Motor interferes with tube at pump inlet. Need offset (gearing system) to avoid this.
2. Aiming to use a standard (straight) PVC tube for the next prototype.
3. Occlusion adjustment is needed. Current roller pumps have this feature.
4. Lubrication needed periodically to reduce friction between the solid cam and silicone tubing to reduce friction.
5. Durability is a concern.
6. The flange 12 to attach the cam to the shaft uses three bolts and requires a large diameter for mounting. Minimizing this diameter in a future prototype will be beneficial.
7. The A shape feature at the end opposing the motor, used to constrain the opposite end of the cam interferes with the tube towards the end of compression. It is believed that this contact creates noise in the pressure vs flow plot.

### Example 4

### Prototype Build and Test: Beta Prototype #2

FIG. 18 shows a rendering of Beta 2 Prototype, opened (solid cam shown). FIG. 19 shows a rendering of Beta 2 Prototype, closed (ball bearing cam shown). Overall dimensions (excluding flat base portion and controller) 101 mm Width×223 mm Length×64 mm Height.

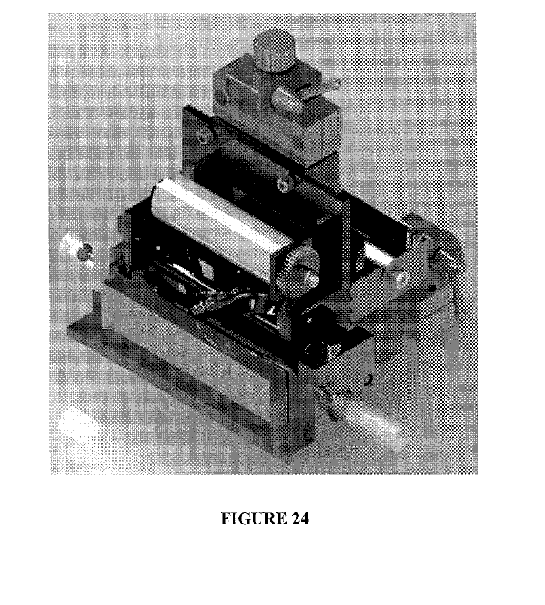

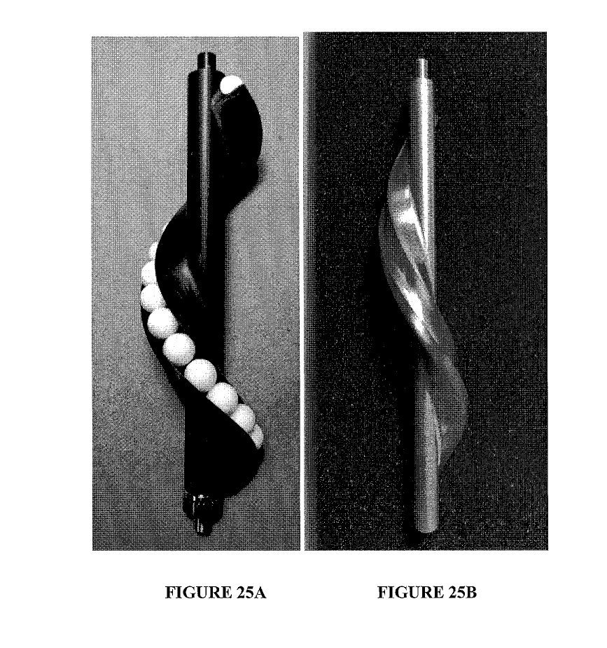

FIG. 24 shows a rendering of Beta 2 Prototype with IGUS linear tables for horizontal and vertical occlusion and motor mounted above the cam. This represents the most current prototype. FIG. 17 illustrates a top level assembly drawing of a beta 2 pump device. Table 1 describes the features of the pump device of FIG. 17. FIG. 21 shows a rendering of the ball bearing cam. This is the mechanism that provides forward flow. FIG. 25A shows an of the ball bearing cam, that was fabricated by Hirsh Precison (Boulder, Colo.) on a 5-axis mill. PTFE ball bearings shown. FIG. 25B shows the machined part before it was annodized.

Description.

The Beta 2 prototype was designed to:
1. Eliminate motor interference with tube at pump inlet.
   a. The Beta prototype mounted the cam concentric to the motor shaft and the diameter of the motor interfered with the tubing slightly, which runs directly underneath the motor as shown in FIG. 24. The motor was mounted to a bracket that compressed the tubing slightly at the inlet.
   b. An offset gearing system was added to this prototype to eliminate this interference.
2. Eliminate the need for a custom tube, allowing standard tubes typically used in a cardiopulmonary bypass circuit.
   a. Added a capturing feature into the base to prevent the tubing from rotating past a certain point as the cam rotates.
   b. Added a feature at the inlet and outlet to provide axial constraint of the tubing.
3. Added both vertical and horizontal occlusion adjustment
   a. Off the shelf linear stage or table can be used
4. Ball bearing cam was machined out of Aluminum, anodized and Teflon coated.
5. A gear (in the 2 gear system mentioned above in 1, b.) is to be pressed onto the cam shaft to reduce the diameter required of the flange system.
6. The bracket system mentioned in 3, a. (above) eliminates the A shape cam constraint used in the Beta 1 prototype.
   Test Circuit: A simple flow circuit was configured, using a venous reservoir provided by the perfusion group. The pump was not set up to augment flow as in previous testing, but rather to provide flow to/from the reservoir.

### Materials:

1. Motor: Maxon DCX32L, 105 W brushless DC motor
   a. Nominal Voltage 24V
   b. Max continuous Torque: 112 mN-m
   c. Stall Torque: 1,980 mN-m
   d. Nominal speed: 7,700 RPM
2. Gearbox: Maxon planetary gearhead GPX32 A
   a. 35:1 gear ratio.
   b. Shaft diameter: 8 mm (with single flat)
   c. Configuration: Maxon motor and gearbox (items 1 and 2) p/n B72E3A119583
   d. Max diameter: 32 mm, length 116.8 mm (includes encoder, excludes shaft)
   e. Max load speed: 220 rev/min
   f. Nominal torque: 3,136 mN-m
   g. Nominal current: 3.82 A
3. Gear System: 0.8 module, 40 tooth gear used on motor shaft. The same size gear was used as a spacer (idler) to space the motor from the cam. A 0.8 module, 20 tooth gear was pressed onto the cam shaft, giving a 2:1 gear ratio, external to the motor gearbox.
4. Additional machined, off the shelf parts and rapid prototype parts were used for construction of the prototype

### Results:

### Flow Results, Beta 2 Prototype

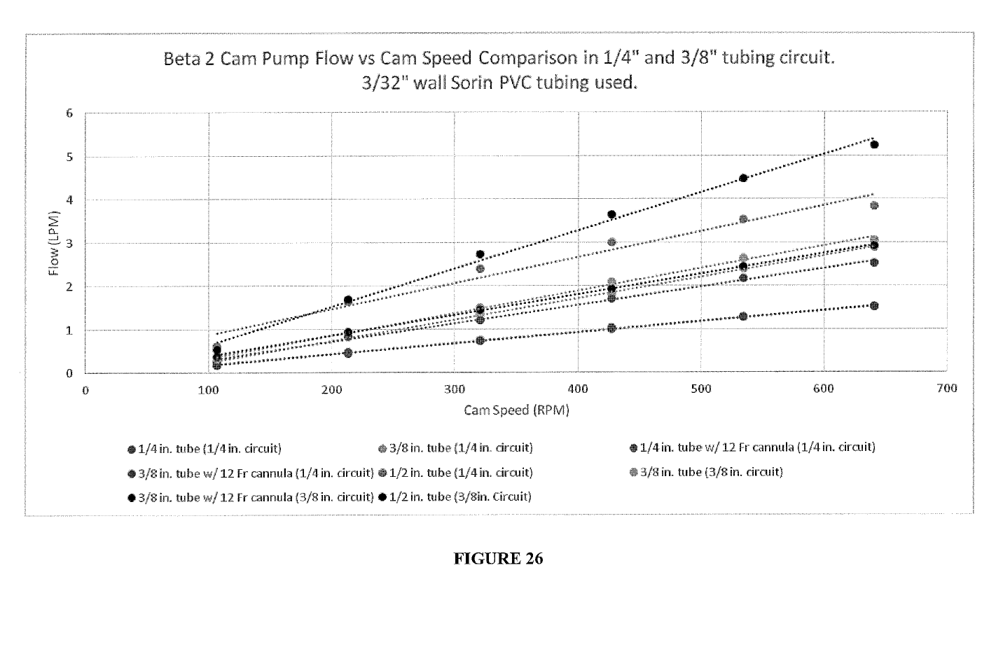

FIG. 26 details results from flow testing in both ¼″ and ⅜″ circuit and through a 12 Fr cannula which increases resistance. Total cam length is 128 mm (26 mm inlet transition, 76 mm full occlusion and 26 mm outlet transition).

### Power Comparison

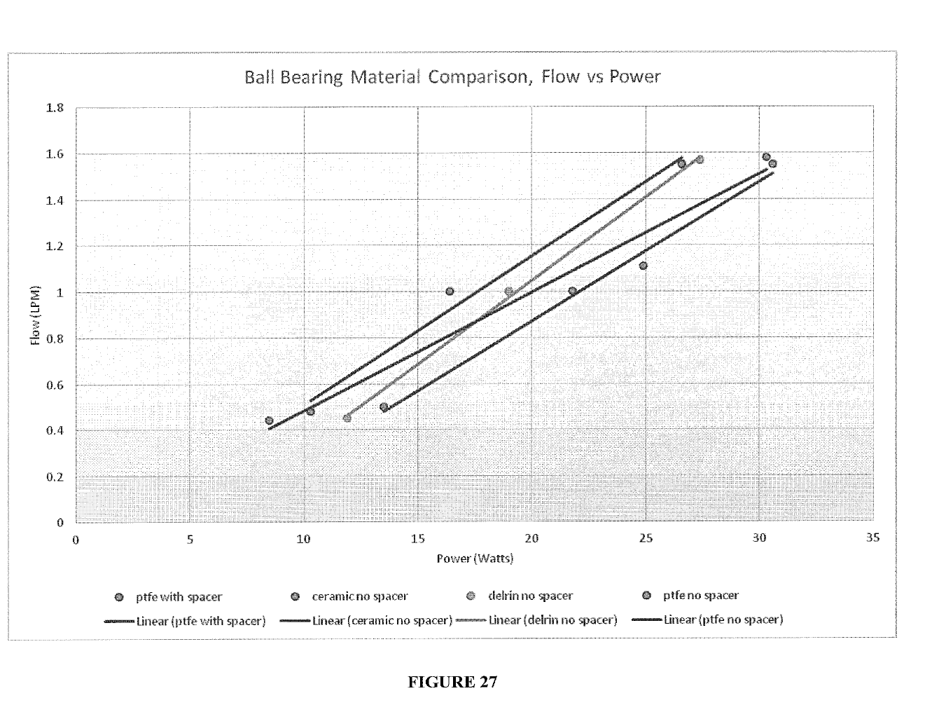

Comparison of power requirements vs flow for different ball bearing materials (ceramic, ptfe and delrin) is shown in FIG. 27. Ceramic ball bearings are optimal of the three materials tested.

### Hemolysis Testing

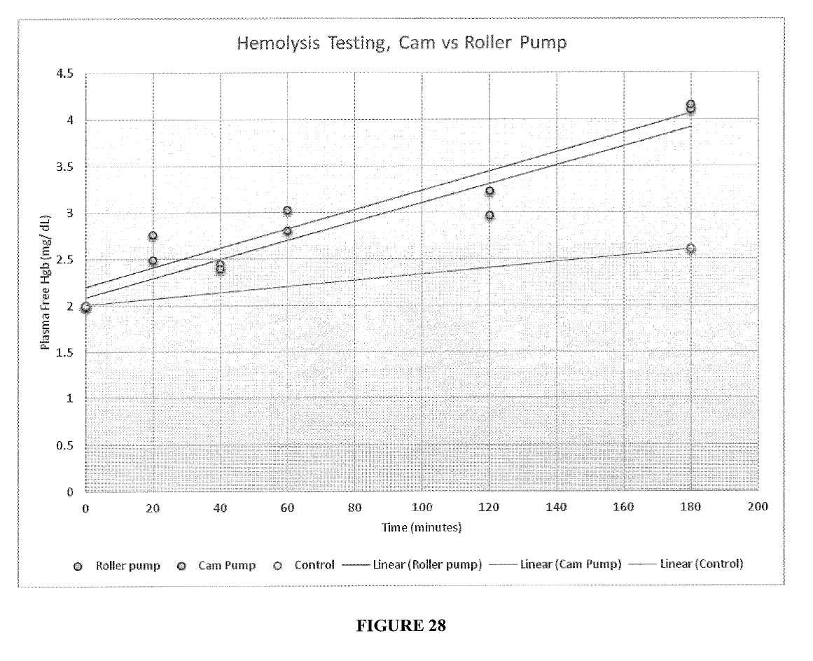

FIG. 28 shows results from hemolysis testing performed on human blood. The cam pump was compared to a roller pump at a flow rate of 1 LPM. A control sample was kept at body temperature and also tested.

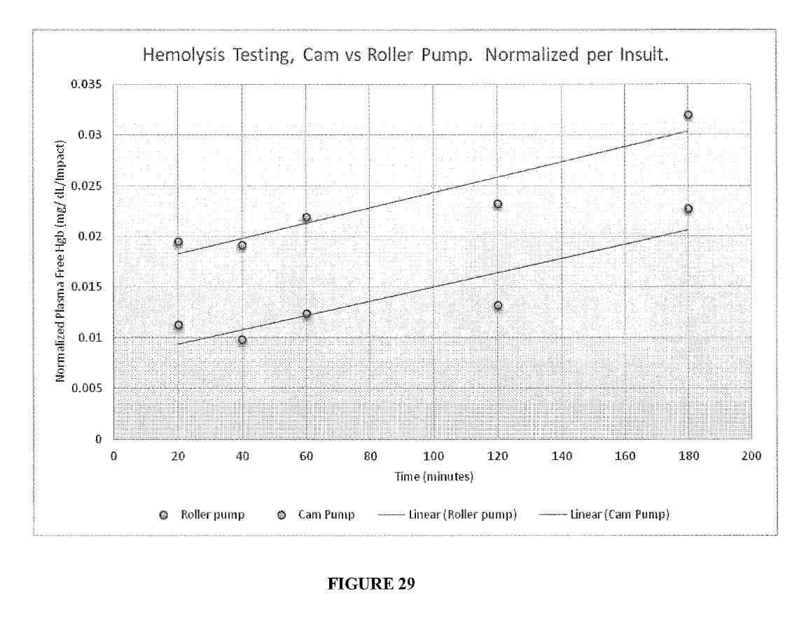

FIG. 29 shows the same hemolysis results that are shown above but normalized per impact (insult). Since the cam tested rotated at a higher speed to achieve an equivalent flow to the 4″ roller pump tested, the results were divided by the number of roller contacts or cam impacts. For the cam tested (76 mm full occlusion length), it rotated at 244 RPM to achieve 1 LPM of flow. The roller pump rotated at 64 RPM and makes two roller impacts per revolution.

### Example 5

### Prototype Build and Test: Future Prototypes

FIG. 30 is a rendering of a portable pump assembly concept that can clamp anywhere on a tube to provide flow.

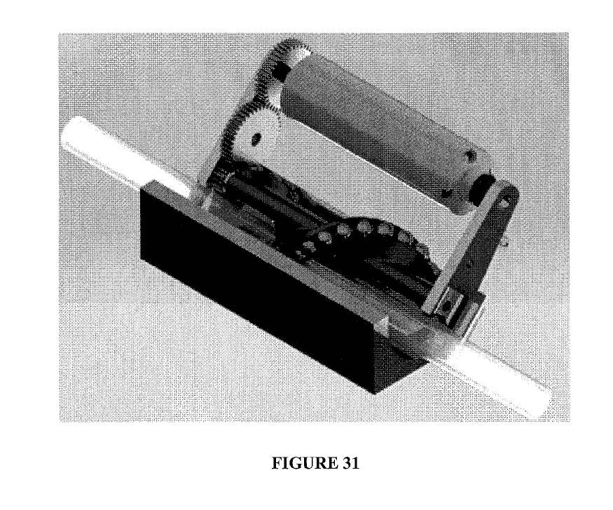

FIG. 31 is a rendering showing the beginning conceptual work for a Beta 3 prototype with one occlusion adjustment for a more compact design.

Thus, specific compositions and methods of pump with external controlled compression and methods of pumping with external controlled compression have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

1. An external cam controlled compression pump comprising:
a) a tube having a first end and a second end in opposition to one another, and a sidewall surrounding a lumen extending between the first end and the second end;
b) a cam having a first end and a second end in opposition to one another, a tube contact surface disposed between the first end and the second end, wherein said cam comprises a helical ridge with at least one axis of rotation, wherein said helical ridge comprises said tube contact surface of said cam;
c) a mount portion configured to rotatably support the cam and compressively position the tube contact surface of the cam in contact with the sidewall of the tube; and
d) a motor in operable connection with the cam and configured to rotate the cam to drive the tube contact surface along the sidewall of the tube from a first location adjacent the first end of the tube to a second location adjacent the second end of the tube.

2. The pump of claim 1 wherein the tube contact surface of the cam is outside of the sidewall of the tube.

3. The pump of claim 1 configured as a device for augmenting blood flow in a single ventricle circuit.

4. The pump of claim 1 further comprising a biocompatible, implantable housing in a surrounding configuration to the tube, cam, and motor.

5. The pump of claim 1 further comprising a biocompatible, implantable housing for intracorporeal implantation.

6. The pump of claim 1 wherein the cam is configured to provide chamber compression within the tube.

7. The pump of claim 1 further comprising an inflow cannula and an outflow cannula in attachment to the first end and the second end of the tube, respectively.

8. The pump of claim 1 further comprising a housing for extracorporeal positioning.

9. The pump of claim 1 wherein the tube is flexible.

10. The pump of claim 1 further comprising a constraint portion in connection with the flexible chamber, the constraint portion providing anchoring with the mount portion.

11. (canceled)

12. The pump of claim 1, wherein said pump comprises at least one type of pump selected from the group consisting of an infusion pump, an oxygenator, a heat exchanger, and a venous reservoir.

13. The pump of claim 1, wherein said pump comprises an implantable infusion pump.

14. (canceled)

15. (canceled)

16. (canceled)

17. (canceled)

18. (canceled)

19. (canceled)

20. (canceled)

21. (canceled)

22. The pump of claim 1 wherein said cam comprises a tapered end.

23. (canceled)

24. (canceled)

25. The pump of claim 19 wherein ridge comprises an attachment feature comprising a friction-reducing element.

26. (canceled)

27. The pump of claim 19 wherein ridge comprises ball bearings.

28. The pump of claim 19 wherein said ridge comprises rollers.

29. The pump of claim 25 wherein said friction-reducing element comprises lubricant.

30. (canceled)

31. (canceled)

32. The pump of claim 1 wherein said cam comprises several linked segments with helical ridges.

33. A method of pumping with external cam controlled compression, the method comprising:
a) providing:
i) an external cam controlled compression pump;
ii) at least one cardiovascular vessel, said vessel comprising fluid;
b) positioning a tube contact surface of a cam in contact with a sidewall of a tube, wherein said tube is connected to said at least one cardiovascular vessel;
c) rotating the cam in contact with the sidewall of the tube to reposition the tube contact surface from a first location adjacent the first end of the tube to a second location adjacent a second end of the tube; and
d) pumping said fluid through said at least one cardiovascular vessel.

34. (canceled)

35. (canceled)

36. (canceled)

37. The method of claim 33 wherein the tube, the cam, and the motor are operable to provide occlusion adjustment.

38-69. (canceled)
